(19) United States
(12) Patent Application Publication
(10) Pub. No.: US 2008/0132551 A1
(43) Pub. Date: Jun. 5, 2008

(54) Positive allosteric modulators of the nicotinic acetylcholine receptor

(75) Inventors: Bruce Nelsen Rogers, Mystic, CT (US); David Walter Piotrowski, Waterford, CT (US); Brandon J. Margolis, Indianapolis, IN (US); Jason K. Myers, Indianapolis, IN (US); Vincent Edward Groppi, Kalamazoo, MI (US); Daniel G. Rudmann, Fishers, IN (US)

Correspondence Address: PFIZER INC;Steve T. Zelson, 150 EAST 42ND STREET, 5TH FLOOR - STOP 49, NEW YORK, NY, 10017-5612, US

(73) Assignee: Pfizer Inc

(21) Appl. No.: 10/808,125

(22) Filed: Mar. 24, 2004

Related U.S. Application Data
(60) Provisional application No. 60/458,766, filed on Mar. 28, 2003.

(51) Int. Cl. A61K 31/4439; C07D 417/02; C07D 413/02; A61K 31/422; A61P 25/28; A61P 25/00; A61K 31/427; A61K 31/433; C07D 285/12

(52) U.S. Cl. 514/342; 546/272.1; 514/340; 546/268.7; 548/146; 514/365; 514/363; 548/136; 514/374; 548/215

(57) ABSTRACT

The invention provides compounds of Formula I:

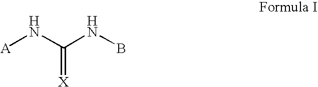

wherein A, B, and X are described herein. These compounds may be in the form of pharmaceutical salts or compositions, may be in pure enantiomeric form or racemic mixtures, and are useful in pharmaceuticals used to treat diseases or conditions in which α7 nAChR is known to be involved.

### CROSS REFERENCE TO RELATED APPLICATIONS

This application claims the benefit of U.S. provisional application Ser. No. 60/458,766 filed on 28 Mar. 2003, under 35 USC 119(e)(i), which is incorporated herein by reference in its entirety.

### FIELD OF INVENTION

This invention relates to the use of certain urea and thiourea compounds as positive allosteric modulators of nicotinic acetylcholine receptors. It also relates to novel urea and thiourea compounds and to pharmaceutical compositions containing them.

### BACKGROUND OF THE INVENTION

Nicotinic acetylcholine receptors (nAChRs) play a large role in central nervous system (CNS) activity and in different tissue throughout the body. They are known to be involved in functions, including, but not limited to, cognition, learning, mood, emotion, and neuroprotection. There are several types of nicotinic acetylcholine receptors, and each one appears to have a different role. Some nicotinic receptors regulate CNS function, including, but not limited to, attention, learning and memory; some regulate pain, inflammation, cancer, and diabetes by controlling tumor necrosis factor alpha (TNF-α); and some regulate vascular angiogenesis; for example, the binding of nicotine to the alpha-7 nAChR stimulates DNA synthesis and proliferation of vascular endothelial cells in vitro (Villablanca, A. C., 1998, J. Appl. Physiol., 84(6):2089-2098) and induces angiogenesis in vivo (Heeschen C., et al. 2002, J. Clin. Invest., 110:527-535; Heeschen, C., et al. 2001, Nature Medicine, 7(7): 833-839). Nicotine affects all such receptors, and has a variety of activities. Unfortunately, not all of the activities are desirable. In fact, undesirable properties of nicotine include its addictive nature and the low ratio between efficacy and safety. The compounds of the present invention activate the α7 nAChR by acting as positive allosteric modulators (PAMs) of this ion channel. These molecules activate the α7 nAChR to enhance the activity of agonists at this receptor, including, but not limited to, acetylcholine (ACh) that is the endogenous neurotransmitter that activates this receptor.

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Both agonist and positive allosteric modulator activity of the α7 nAChR are assayed using a cell-based, calcium flux assay on FLIPR. SHEP-1 cells expressing a novel, mutated form of the α7 nAChR that permitted stable cell surface expression were used for these assays. The details of the mutated form of the α7 nAChR are described in WO 00/73431.

A positive allosteric modulator of α7 nAChR will effectively activate the endogenous α7 nAChR if there is sufficient agonist in the brain and elsewhere within the body to at least partially stimulate this receptor. Therefore, a positive allosteric modulator of α7 nAChR can be administered alone to treat CNS diseases or conditions as discussed herein. In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. In such cases, the positive allosteric modulator of α7 nAChR is administered in combination with another agent that affects the level of agonist.

The activation of the α7 nAChR is also useful to treat, or used to prepare a medicament used to treat, diseases or conditions where a mammal receives symptomatic relief from the decrease of levels of TNF-α. The compounds of the present invention are useful to treat, or are used to prepare a medicament to treat, diseases or conditions where a mammal receives symptomatic relief from the stimulation of vascular angiogenesis.

### SUMMARY OF THE INVENTION

The present invention discloses compounds of the Formula I:

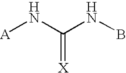

wherein X is O or S;

A is

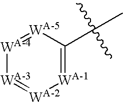

wherein each W^{A-1}, W_{A-2}, W^{A-3}, W^{A-4}, and W^{A-5} are independently N or CR_{A}, provided that no more than four of W^{A-1}, W^{A-2}, W^{A-3}, W_{A-4}, or W^{A-5} are simultaneously N;

Each R^{A} is R_{A-1} or R_{A-2}, provided that one R_{A} is R_{A-2};

Each R_{A-1} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₉, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂;

R_{A-2} is R₁, R₂, OR₁, OR₂, N(R_{A-3})R₁, N(R_{A-3})R₂, SR₁, and SR₂;

R_{A-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

B is a five or six-membered aromatic ring having up to 4 heteroatoms selected from —O—, —N(R_{B-3})—, ═N—, or —S—;

wherein B is

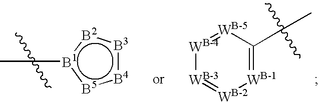

B¹ is N, or C;

B², B³, B⁴, and B are independently N, O, S, C, provided that when valency allows, the N can have a third bond to R_{B-3}, and further provided that when valency allows, the C can have a fourth bond to R_{B-1};

Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂; or

when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N═ and further having substitution where valency allows on the 5-7-membered ring with up to 2 substitutents independently selected from R_{B-2};

Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each W^{B-1}, W^{B-2}, W^{B-3}, W^{B-4}, and W^{B-5} are independently N or CR_{B-1}, provided that no more than 4 of W^{B-1}, W^{B-2}, W^{B-3}, W^{B-4}, or W^{B-5} are simultaneously N;

R₁ is a 5-membered heteroaromatic mono-cyclic moiety containing within the ring 1-3 heteroatoms independently selected from the group consisting of ═N—, —N(R_{1-N})—, —O—, and —S—, and having 0-2 substituent selected from R₁₋₁, and further having 0-4 substituents independently selected from F, Cl, Br, or I;

or R₁ is a 9-membered fused-ring moiety having a 6-membered ring fused to a 5-membered ring including the formula

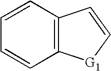

wherein G₁ is O, S or NR_{1-N},

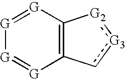

wherein each G is independently CH, C(R_{1-C}), or N, and each G₂ and G₃ are independently selected from CH₂, CH, C(R_{1-C}), O, S, N, and N(R_{1-N}), provided that both G₂ and G₃ are not simultaneously O, simultaneously S, or simultaneously O and S, or

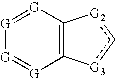

wherein each G is independently CH, C(R_{1-C}), or N, and each G₂ and G₃ are independently selected from CH₂, CH, C(R_{1-C}), O, S, N, and N(R_{1-N}), provided that each 9-membered fused-ring moiety has 0-1 substituent selected from R₁₋₁, and further having 0-3 substituents independently selected from F, Cl, Br, or I, wherein the R₁ moiety attaches to other substituents as defined in formula I at any position as valency allows;

Each R_{1-C} is independently a bond, R₁₋₁, F, Cl, Br, or I, provided that there is only one bond and further provided that R₁ can have only up to one substituent from R₁₋₁, and up to 3 substituents from halogen;

R_{1-N} is H, alkyl, haloalkyl, substituted alkyl, cycloalkyl, halocycloalkyl, substituted cycloalkyl, heterocycloalkyl, haloheterocycloalkyl, or substituted heterocycloalkyl;

R₁₋₁ is alkyl, substituted alkyl, haloalkyl, —OR₁₋₂, —SR₁₋₂, —CN, —NO₂, —N(R₁₋₃)₂;

Each R₁₋₂ is independently H, alkyl, cycloalkyl, heterocycloalkyl, haloalkyl, halocycloalkyl, or haloheterocycloalkyl;

Each R₁₋₃ is independently H, alkyl, cycloalkyl, heterocycloalkyl, haloalkyl, halocycloalkyl, or haloheterocycloalkyl;

R₂ is a 6-membered heteroaromatic mono-cyclic moiety containing within the ring 1-4 heteroatoms selected from ═N— and having 0-1 substituent selected from R₂₋₁ and 0-3 substituent(s) independently selected from F, Cl, Br, or I;

or R₂ is 10-membered heteroaromatic bi-cyclic moieties containing within one or both rings 1-3 heteroatoms selected from ═N—, each 10-membered fused-ring moiety having 0-1 substituent selected from R₂₋₁ and 0-3 substituent(s) independently selected from F, Cl, Br, or I, wherein the R₂ moiety attaches to other substituents as defined in formula I at any position as valency allows;

R₂₋₁ is alkyl, substituted alkyl, haloalkyl, —OR₂₋₂, —SR₂₋₂, —CN, —NO₂, —N(R₂₋₃)₂;

Each R₂₋₂ is independently H, alkyl, cycloalkyl, heterocycloalkyl, haloalkyl, halocycloalkyl, or haloheterocycloalkyl;

Each R₂₋₃ is independently H, alkyl, cycloalkyl, heterocycloalkyl, haloalkyl, halocycloalkyl, or haloheterocycloalkyl;

R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each R₉ is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

or pharmaceutical composition, pharmaceutically acceptable salt, racemic mixture, or pure enantiomer thereof useful to treat any one of or combination of cognitive and attention deficit symptoms of Alzheimer's, neurodegeneration associated with diseases such as Alzheimer's disease, pre-senile dementia (mild cognitive impairment), senile dementia, schizophrenia or psychosis and related associated cognitive deficits, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, symptoms associated with pain; pain and inflammation (rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster); cancer (multiple myeloma; acute and chronic myelogenous leukemia; or cancer-associated cachexia); diabetes (pancreatic beta cell destruction; or type I and type II diabetes); wound healing (healing burns, and wounds in general including from surgery); bone fracture healing; ischemic heart disease, or stable angina pectoris.

Embodiments of the invention may include one or more or combination of the following.

The compounds of Formula I are used to treat, or are used to make a medicament to treat, a mammal where the mammal receives symptomatic relief from activation of an alpha 7 nAChR; these diseases or conditions, include, but are not limited to, any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia or psychosis and related associated cognitive deficits, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain. The compounds of Formula I are also useful to treat or useful to prepare a medicament to treat diseases or conditions where a mammal would receive symptomatic relief from the administration of a compound of Formula I to decrease levels of TNF-α; these diseases or conditions, including, but are not limited to, any one or more or combination of the following: inflammation; pain; cancer; or diabetes. Types of inflammation and/or pain that are to be treated include, but are not limited to, any one or more of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster. Types of cancer that are to be treated include, but are not limited to, any one or more of the following: multiple myeloma; acute and chronic myelogenous leukemia; or cancer-associated cachexia. The compounds of the present invention can be used to treat, or be used to prepare a medicament to treat, the TNF-α aspects associated with pancreatic beta cell destruction; or type I and type II diabetes. The compounds of the present invention are also useful to treat, or to prepare a medicament to be used to treat, diseases or conditions where a mammal would receive symptomatic relief from the increase in vascular angiogenesis; these disease include, but are not limited to, any one or more of the following: wound healing (healing burns, and wounds in general including from surgery), bone fracture healing, ischemic heart disease, and stable angina pectoris.

In another aspect, the invention includes treating, or making medicament(s) to treat, a mammal suffering from schizophrenia or psychosis and cognitive deficits associated with them by administering compounds of Formula I in conjunction with antipsychotic drugs (also called anti-psychotic agents), and optionally also with an agonist of the alpha 7 nAChR, especially when levels of an endogenous agonist are suboptimal. There can be one or more than one medicament. One medicament can comprise the compound of formula I, an antipsychotic agent, and/or an alpha 7 nAChR agonist, or there can be a separate medicament for each separately or any combination, e.g., one medicament could have the compound of Formula I and an alpha 7 nAChR agonist and the other medicament could have the antipsychotic agent.

The compounds of the present invention can also be administered in combination with other agents when treating symptoms associated with infection, inflammation, cancer, or diabetes. For treating these diseases or conditions, a medicament can be prepared comprising a compound of formula I. The same medicament or separate medicament(s), can be used comprising any one of the following: an antibacterial; antiviral agent; an anticancer agent and/or antiemetic agent; or at least one agent to treat diabetes. For example, the compound of Formula I can be co-administered with an antibacterial or antiviral agent, as one medicament or as two separate medicament, to treat an infection, for example, but not limiting, rhinitis. The compound of Formula I can also be co-administered with an anticancer agent and/or antiemetic agent when the disease or condition being treated is cancer, so there could be one medicament or separate medicaments for each agent. And, the compound of Formula I can be co-administered with agents to treat diabetes in one medicament or as separate medicaments.

In a combination therapy, the compounds of Formula I and the other agent(s) can be administered simultaneously or at separate intervals. When administered simultaneously, the compounds of Formula I and the other agent(s) can be incorporated into a single pharmaceutical composition, e.g., a pharmaceutical combination therapy composition. Alternatively, more than one, e.g., two, separate compositions, i.e., one containing a compound of Formula I and the other containing, for example, the psychostimulant, can be administered.

A pharmaceutical combination therapy composition can also be used to treat ADHD, using, for example, but not for limitation, psychostimulants and/or monoamine reuptake inhibitors. This composition can also optionally include an alpha 7 nAChR agonist. While psychostimulants and monoamine reuptake inhibitors control the activity level, and attention, they are not effective in treating the co-morbid or concomitant deficit in cognition that is associated with ADHD. The combination therapy will be more effective at treating this disease because the ability of the mammal to regulate an α7 nAChR agonist will treat the underlying cognitive dysfunction in the disorder and the other two classes of drugs will treat the behavioral problems associated with ADHD. Psychostimulants used for these compositions include, but are not limited to: methylphenidate (Ritalin) administered at about 0.01 to about 0.85 mg/kg/day; dextroamphetamine (Dexedrine) administered at about 0.07 to about 0.85 mg/kg/day; amphetamine (Adderall) administered at about 0.05 to about 0.6 mg/kg/day; and pemoline (Cylert) administered at about 0.1 to about 1.6 mg/kg/day. Monoamine Reuptake inhibitors for these compositions include, but are not limited to: desipramine (Norpramin) administered at about 0.5 to about 5.0 mg/kg/day; nortriptyline administered at about 0.1 to about 3.0 mg/kg/day; atomoxetine (Strattera) administered at about 0.1 to about 3.0 mg/kg/day; reboxetine administered at about 0.03 to about 3.0 mg/kg/day; fluoxetine (Prozac) administered at about 0.2 to about 20 mg/kg/day; tomoxetine administered at about at about 0.1 to about 1.1 mg/kg/day; bupropion (Wellbutrin) administered at about at about 1.0 to about 1.1 mg/kg/day; or modaphonil (Provigil) administered at about at about 1.0 to about 5.7 mg/kg/day. The medicament(s) used to treat ADHD can comprise any combination or single item of the following: a compound of formula I, a psychostimulant, a monoamine reuptake inhibitor and/or an alpha 7 nAChR agonist, or separate medicament(s) can be prepared comprising a any combination of them.

There are also three forms of combination therapies to enhance the activity of a positive allosteric modulator in the presence of an agonist of the α7 nAChR. The first combination therapy is to use a positive allosteric modulator of the α7 nAChR with drugs such as Aricept and Reminyl that inhibit the activity of acetylcholinesterase. Acetylcholinesterase is the enzyme that is primarily responsible for degrading ACh. Drugs such as Aricept and Reminyl which are used to treat Alzheimer's disease, increase ACh levels. The increase in ACh levels leads to an increase in the activity of α7 nAChR and other nicotinic and muscarinic receptors. Thus treating with both acetylcholinesterase inhibitors and a positive allosteric modulator of α7 nAChR will selectively enhance the activity of the α7 nAChR which could provide significant therapeutic benefit for the patient.

The second combination therapy is to use a positive allosteric modulator of the α7 nAChR with a drug that directly activates the α7 nAChR. Drugs that act as receptor agonists and directly activate the α7 nAChR have therapeutic potential but they also carry the liability that prolonged exposure may lead to a loss of efficacy. Using a direct acting agonist of the α7 nAChR in combination with a positive allosteric modulator of the α7 nAChR make both classes of drugs more effective.

The third combination therapy is to use a positive allosteric modulator of α7 nAChR in combination with nutritional supplements including phosphotidylserine, phosphotidylycholine, or choline that act by increasing levels of ACh in the brain. As previously mentioned, an increase in ACh leads to an increase in the activity of α7 nAChR and other nicotinic and muscarinic receptors. Thus, treating with cholinergic nutritional supplements and a positive allosteric modulator of α7 nAChR will selectively enhance the activity of the α7 nAChR to provide significant therapeutic benefit for the patient.

A pharmaceutical combination therapy composition can include therapeutically effective amounts of the compounds of Formula I, and a therapeutically effective amount of the other drug(s)/agent(s). These compositions may be formulated with common excipients, diluents or carriers, and compressed into tablets, or formulated elixirs or solutions for convenient oral administration or administered by intramuscular intravenous routes. The compounds can be administered rectally, topically, orally, or sublingually.

In a combination therapy, the compounds of Formula I and the other drug(s) can be administered simultaneously or at separate intervals. When administered simultaneously the compounds of Formula I and the other drug(s) can be incorporated into a single pharmaceutical composition, e.g., a pharmaceutical combination therapy composition. Alternatively, two or more separate compositions, i.e., one containing compounds of Formula I and the other containing the other drug(s), can be administered simultaneously.

When separately administered, therapeutically effective amounts of compositions containing compounds of Formula I and the other drug(s) are administered on a different schedule. One may be administered before the other as long as the time between the two administrations falls within a therapeutically effective interval. A therapeutically effective interval is a period of time beginning when one of either (a) the compounds of Formula I, or (b) the other drug(s) is administered to a human and ending at the limit of the beneficial effect in the treatment of the disease or condition using the combination of (a) and (b). The methods of administration of the compounds of Formula I and the other drug(s) may vary. Thus, either agent or both agents may be administered rectally, topically, orally, sublingually, or parenterally.

The amount of therapeutically effective compound of Formula I that is administered and the dosage regimen for treating a disease or condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of Formula I. The pharmaceutical compositions may contain the compound of Formula I in the range of about 0.001 to 100 mg/kg/day for an adult, preferably in the range of about 0.01 to about 50 mg/kg/day for an adult. A total daily dose of about 1 to 1000 mg of a compound of Formula I may be appropriate for an adult. The daily dose can be administered in one to four doses per day. These compositions may be formulated with common excipients, diluents or carriers, and compressed into tablets, or formulated elixirs or solutions for convenient oral administration or administered by intramuscular intravenous routes. The compounds of Formula I can be administered rectally, topically, orally, sublingually, or parenterally and maybe formulated as sustained relief dosage forms and the like.

The combined administration of the compounds of Formula I and the other agent(s) is expected to require less of the generally-prescribed dose for either agent when used alone and or is expected to result in less frequent administration of either or both agents. The skilled clinician may in fact learn that behavioral problems are secondary to the cognitive problems and can be treated with lower dosages of the other agent(s). Determining such dosages and routes of administration should be a routine determination by one skilled in the art of treating patients with the diseases or conditions discussed herein.

A group of compounds of Formula I within the scope of the invention includes compounds where X is O or S.

Another group of compounds of Formula I includes compounds where each R_{A} is independently R_{A-1} or R_{A-2}, provided that one R_{A} is R_{A-2}.

Another group of compounds of Formula I includes compounds where each R_{A-1} is independently any one of the following: H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂.

Another group of compounds of Formula I includes compounds where R_{A-2} is any one of the following: R₁, R₂, OR₁, OR₂, N(R_{A-3})R₁, N(R_{A-3})R₂, SR₁, and SR₂.

Another group of compounds of Formula I includes compounds where X is O; A is phenyl substituted at the 2 and 4 position as allowed by Formula I and optionally substituted at the 5 position as allowed by Formula I; and B is independently any one of thienyl, thiazolyl, furanyl, isothiazolyl, thiadiazolyl, isoxazolyl, oxazolyl, and pyridinyl, any of which is optionally substituted as allowed by Formula I, for example with alkyl, haloalkyl, or cyano. More specific examples of A include where W^{A-1} and W^{A-4} are CH; W^{A-2} is CH or CR_{A-1}; W^{A-3} is CR_{A-1}; and W^{A-5} is CR_{A-2}. More specific examples of R_{A-1} include halo or OR₇, where R₇ is alkyl, and substituted alkyl. More specific examples of R_{A-2} include R₁, OR₁, NHR₁, R₂, OR₂, and NHR₂, where R₁ is independently any one of thienyl, thiazolyl, furanyl, isothiazolyl, thiadiazolyl, isoxazolyl, and oxazolyl, and where R₂ is pyridinyl, any of which is optionally substituted as allowed by formula I.

Another group of compounds of Formula I includes compounds where each of R₇, R₈, R₉, and R₁₀ are each independently any one of the following: H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl.

Another group of compounds of Formula I includes compounds where R₅ is alkyl, alkenyl, alkynyl, cycloalkyl, heterocycloalkyl, haloalkyl, haloalkenyl, haloalkynyl, halocycloalkyl, haloheterocycloalkyl, —CN, —NO₂, —OR₃, —SR₃, —N(R₃)₂, —C(O)R₃, —C(O)N(R₃)₂, —NR₃C(O)R₃, —S(O)₂N(R₃)₂, —NR₃S(O)₂R₃, alkyl substituted with 1-4 substituent(s) independently selected from F, Cl, Br, I, or R₆, cycloalkyl substituted with 1-4 substituent(s) independently selected from F, Cl, Br, I, or R₆, or heterocycloalkyl substituted with 1-4 substituent(s) independently selected from F, Cl, Br, I, or R₆.

Another group of compounds of Formula I includes compounds where each R₃ is independently any one of the following: H, alkyl, haloalkyl, alkenyl, haloalkenyl, alkynyl, haloalkynyl, cycloalkyl, halocycloalkyl, heterocycloalkyl, haloheterocycloalkyl, or phenyl optionally substituted with 0-3 halogens and 0-1 substituent selected from alkyl, —CF₃, —CN, —NH₂, —NO₂, and —OH.

Another group of compounds of Formula I includes compounds where R₄ is any one of the following: H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, or aryl.

One of ordinary skill in the art will recognize that where alkyl, haloalkyl and substituted alkyl, alkenyl, haloalkenyl and substituted alkenyl, and the like, are allowed, so is lower alkyl, lower haloalkyl, lower substituted alkyl, lower alkenyl, lower haloalkenyl and lower substituted alkenyl, respectively, are also allowed.

Another group of compounds of Formula I includes compounds where R₆ is any one of the following: —CF₃, —CN, —NO₂, —OR₃, —SR₃, —N(R₃)₂, —C(O)R₃, —C(O)N(R₃)₂, —NR₃C(O)R₃, —S(O)₂N(R₃)₂, or —NR₃S(O)₂R₃.

Non-inclusive examples of R₁ and R₂ include, but are not limited to, any one of the following: thienyl, benzothienyl, pyridyl, thiazolyl, quinolyl, pyrazinyl, pyrimidyl, imidazolyl, furanyl, benzofuranyl, benzothiazolyl, isothiazolyl, thiadiazolyl, benzisothiazolyl, benzisoxazolyl, benzimidazolyl, indolyl, benzoxazolyl, pyrazolyl, triazolyl, isoxazolyl, oxazolyl, pyrrolyl, isoquinolinyl, cinnolinyl, indazolyl, indolizinyl, phthalazinyl, pyridazinyl, triazinyl, isoindolyl, purinyl, oxadiazolyl, furazanyl, benzofurazanyl, benzothiophenyl, benzothiazolyl, quinazolinyl, quinoxalinyl, naphthridinyl, and furopyridinyl, any of which is optionally substituted as allowed by formula I. One of ordinary skill in the art will recognize the moieties from R₁ and R₂ with how they are defined herein. R₁ and R₂ are referred to as heteroaryls for ease of reference.

Another group of compounds of Formula I includes compounds where A includes, but is not limited to, compounds wherein up to four of W^{A-1}, W^{A-2}, W^{A-3}, W^{A-4}, and W^{A-5} can be N to include the following moieties:

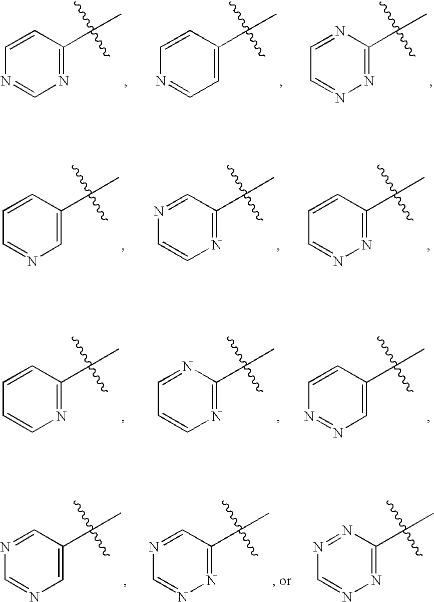

optionally substituted as valency allows and as R_{A} is defined herein.

Another group of compounds of Formula I includes compounds where B includes, but is not limited to, compounds wherein W^{B-1}, W^{B-2}, W^{B-3}, W^{B-4}, and W^{B-5} can be N or CR_{B-1} to include the following moieties:

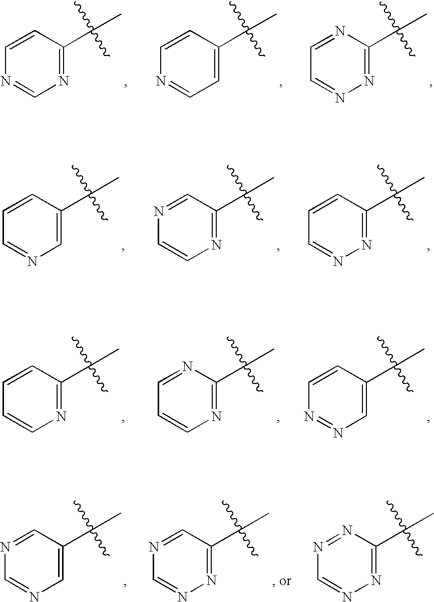

optionally substituted as valency and the definition of Formula I allow and with any definition of R_{B-1} as discussed herein.

Another group of compounds of Formula I includes compounds wherein B includes, but is not limited to, the following moieties that one of ordinary skill in the art can recognize as fitting within the scope of the structures drawn for B:

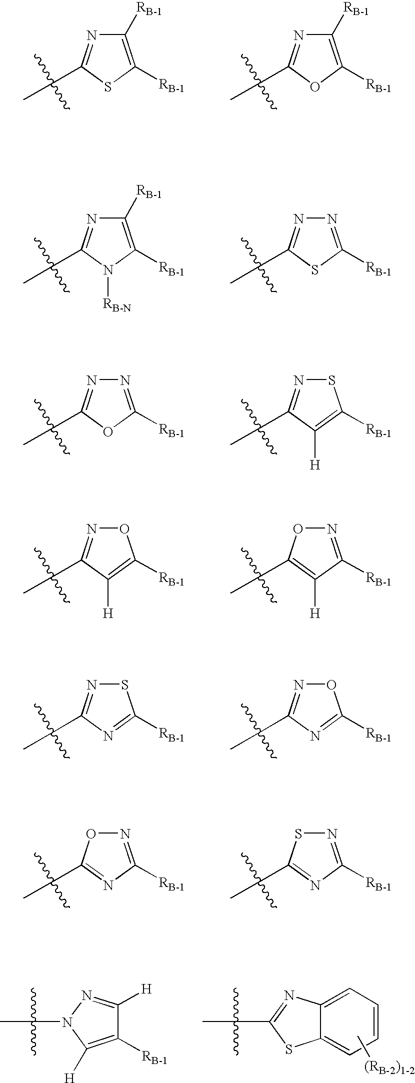

where each R_{B-1} and R_{B-2} have any definition discussed herein and can occur at any carbon where valency allows, and where R_{B-N} has any definition discussed herein and can occur at any nitrogen where valency allows.

The present invention includes, but is not limited to, the following compounds as the free base or a pharmaceutically acceptable salt thereof:
N-[4-ethoxy-2-(pyridin-4-ylamino)phenyl]-N′-(5-methylisoxazol-3-yl)urea;
N-[4-ethoxy-2-(pyridin-3-ylamino)phenyl]-N′-(5-methylisoxazol-3-yl)urea;
N-[4-ethoxy-2-(pyridin-3-ylamino)phenyl]-N′-[5-(trifluoromethyl)-1,3,4-thiadiazol-2-yl]urea;
N-[4-ethoxy-2-(pyridin-2-ylamino)phenyl]-N′-(5-methylisoxazol-3-yl)urea;
N-[4-methoxy-2-(1,3-thiazol-2-yl)phenyl]-N′-(5-methylisoxazol-3-yl)urea;
N-[4-methoxy-2-(1,3-thiazol-2-yl)phenyl]-N′-[5-(trifluoromethyl)-1,3,4-thiadiazol-2-yl]urea;
N-[4-methoxy-2-(1,3-oxazol-2-yl)phenyl]-N′-(5-methylisoxazol-3-yl)urea;
N-[4-methoxy-2-(1,3-oxazol-2-yl)phenyl]-N′-[5-(trifluoromethyl)-1,3,4-thiadiazol-2-yl]urea;
N-[2-(2-furyl)-4-methoxyphenyl]-N′-[5-(trifluoromethyl)-1,3,4-thiadiazol-2-yl]urea;
N-[2-(2-furyl)-4-methoxyphenyl]-N′-[3-(trifluoromethyl)isoxazol-5-yl]urea;
N-[4-ethoxy-2-(2-furyl)phenyl]-N′-[5-(trifluoromethyl)-1,3,4-thiadiazol-2-yl]urea;
N-[4-ethoxy-2-(2-furyl)phenyl]-N′-(5-methylisoxazol-3-yl)urea;
N-(4-methoxy-2-thien-2-ylphenyl)-N′-[5-(trifluoromethyl)-1,3,4-thiadiazol-2-yl]urea;
N-[2,4-dimethoxy-5-(1,3-oxazol-2-yl)phenyl]-N′-[5-(trifluoromethyl)-1,3,4-thiadiazol-2-yl]urea;
N-[4-ethoxy-2-(2-furyl)phenyl]-N′-[3-(trifluoromethyl)isoxazol-5-yl]urea;
N-[4-methoxy-2-(1,3-oxazol-2-yl)phenyl]-N′-[3-(trifluoromethyl)isoxazol-5-yl]urea;
N-[4-ethoxy-2-(1,3-oxazol-2-yl)phenyl]-N′-[3-(trifluoromethyl)isoxazol-5-yl]urea;
N-[4-ethoxy-2-(1,3-thiazol-2-yl)phenyl]-N′-[3-(trifluoromethyl)isoxazol-5-yl]urea;
N-[4-ethoxy-2-(1,3-oxazol-2-yl)phenyl]-N′-[5-(trifluoromethyl)-1,3,4-thiadiazol-2-yl]urea;
N-[4-ethoxy-2-(1,3-thiazol-2-yl)phenyl]-N′-[5-(trifluoromethyl)-1,3,4-thiadiazol-2-yl]urea;
N-(6-cyanopyridin-3-yl)-N′-[4-ethoxy-2-(1,3-oxazol-2-yl)phenyl]urea;
N-[2-(1,3-oxazol-2-yl)phenyl]-N′-[3-(trifluoromethyl)isoxazol-5-yl]urea;
N-[2-(2-furyl)phenyl]-N′-[5-(trifluoromethyl)-1,3,4-thiadiazol-2-yl]urea;
N-[4-ethoxy-2-(2-furyl)phenyl]-N′-[5-(trifluoromethyl)isoxazol-3-yl]urea; and
N-[4-ethoxy-2-(1,3-oxazol-2-yl)phenyl]-N′-[5-(trifluoromethyl)isoxazol-3-yl]urea.

The present invention includes, but is not limited to, the following compounds as the free base or a pharmaceutically acceptable salt thereof:
N-[4-ethoxy-5-fluoro-2-(pyridin-4-ylamino)phenyl]-N′-(5-methylisoxazol-3-yl)urea;
N-[4-ethoxy-5-fluoro-2-(pyridin-3-ylamino)phenyl]-N′-(5-methylisoxazol-3-yl)urea;
N-[4-ethoxy-5-fluoro-2-(pyridin-3-ylamino)phenyl]-N′-[5-(trifluoromethyl)-1,3,4-thiadiazol-2-yl]urea;
N-[4-ethoxy-5-fluoro-2-(pyridin-2-ylamino)phenyl]-N′-(5-methylisoxazol-3-yl)urea;
N-[4-methoxy-5-fluoro-2-(1,3-thiazol-2-yl)phenyl]-N′-(5-methylisoxazol-3-yl)urea;
N-[4-methoxy-5-fluoro-2-(1,3-thiazol-2-yl)phenyl]-N′-[5-(trifluoromethyl)-1,3,4-thiadiazol-2-yl]urea;
N-[4-methoxy-5-fluoro-2-(1,3-oxazol-2-yl)phenyl]-N′-(5-methylisoxazol-3-yl)urea;
N-[4-methoxy-5-fluoro-2-(1,3-oxazol-2-yl)phenyl]-N′-[5-(trifluoromethyl)-1,3,4-thiadiazol-2-yl]urea;
N-[5-fluoro-2-(2-furyl)-4-methoxyphenyl]-N′-[5-(trifluoromethyl)-1,3,4-thiadiazol-2-yl]urea;
N-[5-fluoro-2-(2-furyl)-4-methoxyphenyl]-N′-[3-(trifluoromethyl)isoxazol-5-yl]urea;
N-[4-ethoxy-5-fluoro-2-(2-furyl)phenyl]-N′-[5-(trifluoromethyl)-1,3,4-thiadiazol-2-yl]urea;
N-[4-ethoxy-5-fluoro-2-(2-furyl)phenyl]-N′-(5-methylisoxazol-3-yl)urea;
N-(5-fluoro-4-methoxy-2-thien-2-ylphenyl)-N′-[5-(trifluoromethyl)-1,3,4-thiadiazol-2-yl]urea;
N-[4-ethoxy-5-fluoro-2-(2-furyl)phenyl]-N′-[3-(trifluoromethyl)isoxazol-5-yl]urea;
N-[4-methoxy-5-fluoro-2-(1,3-oxazol-2-yl)phenyl]-N′-[3-(trifluoromethyl)isoxazol-5-yl]urea;
N-[4-ethoxy-5-fluoro-2-(1,3-oxazol-2-yl)phenyl]-N′-[3-(trifluoromethyl)isoxazol-5-yl]urea;
N-[4-ethoxy-5-fluoro-2-(1,3-thiazol-2-yl)phenyl]-N′-[3-(trifluoromethyl)isoxazol-5-yl]urea;
N-[4-ethoxy-5-fluoro-2-(1,3-oxazol-2-yl)phenyl]-N′-[5-(trifluoromethyl)-1,3,4-thiadiazol-2-yl]urea;
N-[4-ethoxy-5-fluoro-2-(1,3-thiazol-2-yl)phenyl]-N′-[5-(trifluoromethyl)-1,3,4-thiadiazol-2-yl]urea;
N-(6-cyanopyridin-3-yl)-N′-[4-ethoxy-5-fluoro-2-(1,3-oxazol-2-yl)phenyl]urea;
N-[5-fluoro-2-(1,3-oxazol-2-yl)phenyl]-N′-[3-(trifluoromethyl)isoxazol-5-yl]urea;
N-[5-fluoro-2-(2-furyl)phenyl]-N′-[5-(trifluoromethyl)-1,3,4-thiadiazol-2-yl]urea;
N-[4-ethoxy-5-fluoro-2-(2-furyl)phenyl]-N′-[5-(trifluoromethyl)isoxazol-3-yl]urea;
N-[4-ethoxy-5-fluoro-2-(1,3-oxazol-2-yl)phenyl]-N′-[5-(trifluoromethyl)isoxazol-3-yl]urea;
N-[5-chloro-4-ethoxy-2-(pyridin-4-ylamino)phenyl]-N′-(5-methylisoxazol-3-yl)urea;
N-[5-chloro-4-ethoxy-2-(pyridin-3-ylamino)phenyl]-N′-(5-methylisoxazol-3-yl)urea;
N-[5-chloro-4-ethoxy-2-(pyridin-3-ylamino)phenyl]-N′-[5-(trifluoromethyl)-1,3,4-thiadiazol-2-yl]urea;
N-[5-chloro-4-ethoxy-2-(pyridin-2-ylamino)phenyl]-N′-(5-methylisoxazol-3-yl)urea;
N-[5-chloro-4-methoxy-2-(1,3-thiazol-2-yl)phenyl]-N′-(5-methylisoxazol-3-yl)urea;
N-[5-chloro-4-methoxy-2-(1,3-thiazol-2-yl)phenyl]-N′-[5-(trifluoromethyl)-1,3,4-thiadiazol-2-yl]urea;
N-[5-chloro-4-methoxy-2-(1,3-oxazol-2-yl)phenyl]-N′-(5-methylisoxazol-3-yl)urea;
N-[5-chloro-4-methoxy-2-(1,3-oxazol-2-yl)phenyl]-N′-[5-(trifluoromethyl)-1,3,4-thiadiazol-2-yl]urea;
N-[5-chloro-2-(2-furyl)-4-methoxyphenyl]-N′-[5-(trifluoromethyl)-1,3,4-thiadiazol-2-yl]urea;
N-[5-chloro-2-(2-furyl)-4-methoxyphenyl]-N′-[3-(trifluoromethyl)isoxazol-5-yl]urea;
N-[5-chloro-4-ethoxy-2-(2-furyl)phenyl]-N′-[5-(trifluoromethyl)-1,3,4-thiadiazol-2-yl]urea;
N-[5-chloro-4-ethoxy-2-(2-furyl)phenyl]-N′-(5-methylisoxazol-3-yl)urea;
N-(5-chloro-4-methoxy-2-thien-2-ylphenyl)-N′-[5-(trifluoromethyl)-1,3,4-thiadiazol-2-yl]urea;
N-[5-chloro-4-ethoxy-2-(2-furyl)phenyl]-N′-[3-(trifluoromethyl)isoxazol-5-yl]urea;
N-[5-chloro-4-methoxy-2-(1,3-oxazol-2-yl)phenyl]-N′-[3-(trifluoromethyl)isoxazol-5-yl]urea;
N-[5-chloro-4-ethoxy-2-(1,3-oxazol-2-yl)phenyl]-N′-[3-(trifluoromethyl)isoxazol-5-yl]urea;
N-[5-chloro-4-ethoxy-2-(1,3-thiazol-2-yl)phenyl]-N′-[3-(trifluoromethyl)isoxazol-5-yl]urea;
N-[5-chloro-4-ethoxy-2-(1,3-oxazol-2-yl)phenyl]-N′-[5-(trifluoromethyl)-1,3,4-thiadiazol-2-yl]urea;
N-[5-chloro-4-ethoxy-2-(1,3-thiazol-2-yl)phenyl]-N′-[5-(trifluoromethyl)-1,3,4-thiadiazol-2-yl]urea;
N-(6-cyanopyridin-3-yl)-N′-[5-chloro-4-ethoxy-2-(1,3-oxazol-2-yl)phenyl]urea;
N-[5-chloro-2-(1,3-oxazol-2-yl)phenyl]-N′-[3-(trifluoromethyl)isoxazol-5-yl]urea;
N-[5-chloro-2-(2-furyl)phenyl]-N′-[5-(trifluoromethyl)-1,3,4-thiadiazol-2-yl]urea;
N-[5-chloro-4-ethoxy-2-(2-furyl)phenyl]-N′-[5-(trifluoromethyl)isoxazol-3-yl]urea;
N-[5-chloro-4-ethoxy-2-(1,3-oxazol-2-yl)phenyl]-N′-[5-(trifluoromethyl)isoxazol-3-yl]urea;
N-[4-(2-methoxy-ethoxy)-2-(pyridin-4-ylamino)phenyl]-N′-(5-methylisoxazol-3-yl)urea;
N-[4-(2-methoxy-ethoxy)-2-(pyridin-3-ylamino)phenyl]-N′-(5-methylisoxazol-3-yl)urea;
N-[4-(2-methoxy-ethoxy)-2-(pyridin-3-ylamino)phenyl]-N′-[5-(trifluoromethyl)-1,3,4-thiadiazol-2-yl]urea;
N-[4-(2-methoxy-ethoxy)-2-(pyridin-2-ylamino)phenyl]-N′-(5-methylisoxazol-3-yl)urea;
N-[4-(2-methoxy-ethoxy)-2-(2-furyl)phenyl]-N′-[5-(trifluoromethyl)-1,3,4-thiadiazol-2-yl]urea;
N-[4-(2-methoxy-ethoxy)-2-(2-furyl)phenyl]-N′-(5-methylisoxazol-3-yl)urea;
N-[4-(2-methoxy-ethoxy)-2-(2-furyl)phenyl]-N′-[3-(trifluoromethyl)isoxazol-5-yl]urea;
N-[4-(2-methoxy-ethoxy)-2-(1,3-oxazol-2-yl)phenyl]-N′-[3-(trifluoromethyl)isoxazol-5-yl]urea;
N-[4-(2-methoxy-ethoxy)-2-(1,3-thiazol-2-yl)phenyl]-N′-[3-(trifluoromethyl)isoxazol-5-yl]urea;
N-[4-(2-methoxy-ethoxy)-2-(1,3-oxazol-2-yl)phenyl]-N′-[5-(trifluoromethyl)-1,3,4-thiadiazol-2-yl]urea;
N-[4-(2-methoxy-ethoxy)-2-(1,3-thiazol-2-yl)phenyl]-N′-[5-(trifluoromethyl)-1,3,4-thiadiazol-2-yl]urea;
N-(6-cyanopyridin-3-yl)-N′-[4-(2-methoxy-ethoxy)-2-(1,3-oxazol-2-yl)phenyl]urea;
N-[4-(2-methoxy-ethoxy)-2-(2-furyl)phenyl]-N′-[5-(trifluoromethyl)isoxazol-3-yl]urea;
N-[4-(2-methoxy-ethoxy)-2-(1,3-oxazol-2-yl)phenyl]-N′-[5-(trifluoromethyl)isoxazol-3-yl]urea;
N-[5-fluoro-4-(2-methoxy-ethoxy)-2-(pyridin-4-ylamino)phenyl]-N′-(5-methylisoxazol-3-yl)urea;
N-[5-fluoro-4-(2-methoxy-ethoxy)-2-(pyridin-3-ylamino)phenyl]-N′-(5-methylisoxazol-3-yl)urea;
N-[5-fluoro-4-(2-methoxy-ethoxy)-2-(pyridin-3-ylamino)phenyl]-N′-[5-(trifluoromethyl)-1,3,4-thiadiazol-2-yl]urea;
N-[5-fluoro-4-(2-methoxy-ethoxy)-2-(pyridin-2-ylamino)phenyl]-N′-(5-methylisoxazol-3-yl)urea;
N-[5-fluoro-4-(2-methoxy-ethoxy)-2-(2-furyl)phenyl]-N′-[5-(trifluoromethyl)-1,3,4-thiadiazol-2-yl]urea;
N-[5-fluoro-4-(2-methoxy-ethoxy)-2-(2-furyl)phenyl]-N′-(5-methylisoxazol-3-yl)urea;
N-[5-fluoro-4-(2-methoxy-ethoxy)-2-(2-furyl)phenyl]-N′-[3-(trifluoromethyl)isoxazol-5-yl]urea;
N-[5-fluoro-4-(2-methoxy-ethoxy)-2-(1,3-oxazol-2-yl)phenyl]-N′-[3-(trifluoromethyl)isoxazol-5-yl]urea;
N-[5-fluoro-4-(2-methoxy-ethoxy)-2-(1,3-thiazol-2-yl)phenyl]-N′-[3-(trifluoromethyl)isoxazol-5-yl]urea;
N-[5-fluoro-4-(2-methoxy-ethoxy)-2-(1,3-oxazol-2-yl)phenyl]-N′-[5-(trifluoromethyl)-1,3,4-thiadiazol-2-yl]urea;
N-[5-fluoro-4-(2-methoxy-ethoxy)-2-(1,3-thiazol-2-yl)phenyl]-N′-[5-(trifluoromethyl)-1,3,4-thiadiazol-2-yl]urea;
N-(6-cyanopyridin-3-yl)-N′-[5-fluoro-4-(2-methoxy-ethoxy)-2-(1,3-oxazol-2-yl)phenyl]urea;
N-[5-fluoro-4-(2-methoxy-ethoxy)-2-(2-furyl)phenyl]-N′-[5-(trifluoromethyl)isoxazol-3-yl]urea; and
N-[5-fluoro-4-(2-methoxy-ethoxy)-2-(1,3-oxazol-2-yl)phenyl]-N′-[5-(trifluoromethyl)isoxazol-3-yl]urea.

The present invention also includes isotopically labeled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine iodine, and chlorine, such as ²H, ³H, ¹³C, ¹¹C, ¹⁴C, ¹⁵N, ¹⁸O, ¹⁷O, ³¹P, ³²P, ³⁵S, ¹⁸F, ¹²³I, and ³⁶C, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances.

Isotopically labeled compounds of Formula I can generally be prepared by carrying out the synthetic procedures described herein by substituting an isotopically labeled reagent for a non-isotopically labeled reagent. Isotopically labeled reagents are described, for example, by Langstrom in Acta Chem. Scand. S37: 147 (1990). Introducing ¹¹C-labeled agonists of nAChR has been described in Dolle, Frederic, et al, J. Labelled Cps Radiopharm., 2001; 44: 785-795. For a general discussion of nuclear imaging, see, “Nuclear Imaging in Drug Discovery, Development, and Approval, H. D. Burns, et al. (Eds).

The present invention also includes compounds for use in photoaffinity labeling experiments. One technique for the biochemical characterization of receptors is photoaffinity labeling using a photolabile molecule, or probe, which binds with high affinity to a receptor and can be irreversibly incorporated into the receptor under the influence of ultraviolet light. In order to have an effective and useful photoaffinity probe, several requirements must be met. First, the probe must have good biological activity at the same target protein relative to the parent compounds of interest. Second, it must have a reactive group which can covalently bond to the target site upon photoactivation. For example, the azido group is chemically inert until photoactivated by UV light. Upon photolysis it generates a highly reactive nitrene which inserts into either the peptide backbone or the amino acid side chains of the protein to which it is bound. This insertion forms a covalent linkage between the photoprobe and the protein allowing it to be permanently tagged for identification.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF INVENTION

Surprisingly, we have found that compounds of Formula I:

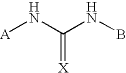

wherein X is O or S;

A is

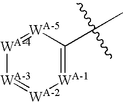

wherein each W^{A-1}, W^{A-2}, W^{A-3}, W^{A-4}, and W^{A-5} are independently N or CR_{A}, provided that no more than four of W^{A-1}, W^{A-2}, W^{A-3}, W^{A-4}, or W^{A-5} are simultaneously N;

Each R_{A} is R_{A-1} or R_{A-2}, provided that one R_{A} is R_{A-2};

Each R_{A-1} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂;

R_{A-2} is R₁, R₂, OR₁, OR₂, N(R_{A-3})R₁, N(R_{A-3})R₂, SR₁, and SR₂;

R_{A-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

B is a five or six-membered aromatic ring having up to 4 heteroatoms selected from —O—, —N(R_{B-3})—, ═N—, or —S—;

wherein B is

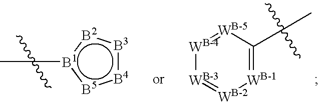

B¹ is N, or C;

B², B³, B⁴, and B⁵ are independently N, O, S, C, provided that when valency allows, the N can have a third bond to R_{B-3}, and further provided that when valency allows, the C can have a fourth bond to R_{B-1};

Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂; or

when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N═ and further having substitution where valency allows on the 5-7-membered ring with up to 2 substitutents independently selected from R_{B-2};

Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;

R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each W^{B-1}, W^{B-2}, W^{B-3}, W^{B-4}, and W^{B-5} are independently N or CR_{B-1}, provided that no more than 4 of W^{B-1}, W^{B-2}, W^{B-3}, W^{B-4}, or W^{B-5} are simultaneously N;

Halogen (used interchangeably with “halo”) is F, Br, Cl, or I;

Alkyl is both straight- and branched-chain moieties having from 1-6 carbon atoms;

Lower alkyl is both straight- and branched-chain moieties having from 1-4 carbon atoms;

Haloalkyl is an alkyl moiety having from 1-6 carbon atoms and having 1 to (2n+1) substituent(s) independently selected from F, Cl, Br, or I, where n is the maximum number of carbon atoms in the moiety;

Lower haloalkyl is lower alkyl having 1 to (2n+1) substituent(s) independently selected from F, Cl, Br, or I, where n is the maximum number of carbon atoms in the moiety;

Substituted alkyl is an alkyl moiety from 1-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃(O)₂R₃, phenyl, or substituted phenyl;

Lower substituted alkyl is lower alkyl having 0-3 substituents independently selected from F, Cl, Br, or I, and further having 1 substituent selected from —CN, —NO₂, —OR₃—SR₃, —N(R₃)₂, —C(O)R₃—C(O)N(R₃)₂, —NR₃(O)R₃—S(O)₂N(R₃)₂, —NR₃(O)₂R₃, phenyl, or substituted phenyl;

Alkenyl is straight- and branched-chain moieties having from 2-6 carbon atoms and having at least one carbon-carbon double bond;

Lower alkenyl is straight- and branched-chain moieties having from 2-4 carbon atoms and having at least one carbon-carbon double bond;

Haloalkenyl is an alkenyl moiety having from 2-6 carbon atoms and having 1 to (2n−1) substituent(s) independently selected from F, Cl, Br, or I where n is the maximum number of carbon atoms in the moiety;

Lower haloalkenyl is lower alkenyl having 1 to (2n−1) substituent(s) independently selected from F, Cl, Br, or I where n is the maximum number of carbon atoms in the moiety;

Substituted alkenyl is an unsaturated alkenyl moiety having from 2-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Lower substituted alkenyl is lower alkenyl having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Alkynyl is straight- and branched-chained moieties having from 2-6 carbon atoms and having at least one carbon-carbon triple bond;

Haloalkynyl is an alkynyl moiety having from 2-6 carbon atoms and having 1 to (2n−3) substituent(s) independently selected from F, Cl, Br, or I where n is the maximum number of carbon atoms in the moiety;

Substituted alkynyl is an unsaturated alkynyl moiety having from 2-6 carbon atoms and having 0-3 substituents independently selected from F, Cl, Br, or I, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Cycloalkyl is a cyclic alkyl moiety having from 3-6 carbon atoms;

Lower cycloalkyl is a cyclic alkyl moiety having from 3-4 carbon atoms;

Halocycloalkyl is a cyclic moiety having from 3-6 carbon atoms and having 1-4 substituents independently selected from F, Cl, Br, or I;

Substituted cycloalkyl is a cycloalkyl moiety from 3-6 carbon atoms and having 0-3 substituents independently selected from F, Cl, Br, or I and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Heterocycloalkyl is a cyclic moiety having 4-7 atoms with 1-2 atoms within the ring being —S—, —N(R₄)—, or —O—;

Haloheterocycloalkyl is a cyclic moiety having from 4-7 atoms with 1-2 atoms within the ring being —S—, —N(R₄)—, or —O—, and having 1-4 substituents independently selected from F, Br, Cl, or I;

Substituted heterocycloalkyl is a cyclic moiety having from 4-7 atoms with 1-2 atoms within the ring being —S—, —N(R₄)—, or —O— and having 0-3 substituents independently selected from F, Br, Cl, or I, further having up to 2 oxo (═O) on separate carbon atoms with sufficient valency, and further having 1 substituent selected from —CN, —NO₂, —OR₃, —SR₃, —N(R₃)₂, —C(O)R₃, —C(O)N(R₃)₂, —NR₃C(O)R₃, —S(O)₂N(R₃)₂, —NR₃C(O)₂R₃, phenyl, or substituted phenyl;

Aryl is phenyl, substituted phenyl, naphthyl, or substituted naphthyl;

Substituted phenyl is a phenyl either having 1-4 substituents independently selected from F, Cl, Br, or I, or having 1 substituent selected from R₅ and 0-3 substituents independently selected from F, Cl, Br, or I;

Substituted naphthyl is a naphthalene moiety either having 1-4 substituents independently selected from F, Cl, Br, or I, or having 1 substituent selected from R₅ and 0-3 substituents independently selected from F, Cl, Br, or I, where the substitution can be independently on either only one ring or both rings of said naphthalene moiety;

R₁ is a 5-membered heteroaromatic mono-cyclic moiety containing within the ring 1-3 heteroatoms independently selected from the group consisting of ═N—, —N(R_{1-N})—, —O—, and —S—, and having 0-2 substituent selected from R₁₋₁, and further having 0-4 substituents independently selected from F, Cl, Br, or I;

or R₁ is a 9-membered fused-ring moiety having a 6-membered ring fused to a 5-membered ring including the formula

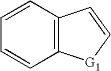

wherein G₁ is O, S or NR_{1-N},

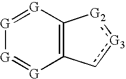

wherein each G is independently CH, C(R_{1-C}), or N, and each G₂ and G₃ are independently selected from CH₂, CH, C(R_{1-C}), O, S, N, and N(R_{1-N}), provided that both G₂ and G₃ are not simultaneously O, simultaneously S, or simultaneously O and S, or

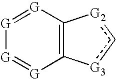

wherein each G is independently CH, C(R_{1-C}), or N, and each G₂ and G₃ are independently selected from CH₂, CH, C(R_{1-C}), O, S, N, and N(R_{1-N}), provided that each 9-membered fused-ring moiety has 0-1 substituent selected from R₁₋₁, and further having 0-3 substituents independently selected from F, Cl, Br, or I, wherein the R₁ moiety attaches to other substituents as defined in formula I at any position as valency allows;

Each R_{1-C} is independently a bond, R₁₋₁, F, Cl, Br, or I, provided that there is only one bond and further provided that R₁ can have only up to one substituent from R₁₋₁, and up to 3 substituents from halogen;

R_{1-N} is H, alkyl, haloalkyl, substituted alkyl, cycloalkyl, halocycloalkyl, substituted cycloalkyl, heterocycloalkyl, haloheterocycloalkyl, or substituted heterocycloalkyl;

R₁₋₁ is alkyl, substituted alkyl, haloalkyl, —OR₁₋₂, —SR₁₋₂, —CN, —NO₂, —N(R₁₋₃)₂;

Each R₁₋₂ is independently H, alkyl, cycloalkyl, heterocycloalkyl, haloalkyl, halocycloalkyl, or haloheterocycloalkyl;

Each R₁₋₃ is independently H, alkyl, cycloalkyl, heterocycloalkyl, haloalkyl, halocycloalkyl, or haloheterocycloalkyl;

R₂ is a 6-membered heteroaromatic mono-cyclic moiety containing within the ring 1-4 heteroatoms selected from ═N— and having 0-1 substituent selected from R₂₋₁ and 0-3 substituent(s) independently selected from F, Cl, Br, or I;

or R₂ is 10-membered heteroaromatic bi-cyclic moieties containing within one or both rings 1-3 heteroatoms selected from ═N—, each 10-membered fused-ring moiety having 0-1 substituent selected from R₂₋₁ and 0-3 substituent(s) independently selected from F, Cl, Br, or I, wherein the R₂ moiety attaches to other substituents as defined in formula I at any position as valency allows;

R₂₋₁ is alkyl, substituted alkyl, haloalkyl, —OR₂₋₂, —SR₂₋₂, —CN, —NO₂, —N(R₂₋₃)₂;

Each R₂₋₂ is independently H, alkyl, cycloalkyl, heterocycloalkyl, haloalkyl, halocycloalkyl, or haloheterocycloalkyl;

Each R₂₋₃ is independently H, alkyl, cycloalkyl, heterocycloalkyl, haloalkyl, halocycloalkyl, or haloheterocycloalkyl;

Each R₃ is independently H, alkyl, haloalkyl, alkenyl, haloalkenyl, alkynyl, haloalkynyl, cycloalkyl, halocycloalkyl, heterocycloalkyl, haloheterocycloalkyl, or phenyl optionally substituted with 0-3 halogens and 0-1 substituent selected from alkyl, —CF₃, —CN, —NH₂, —NO₂, and —OH;

R₄ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, or aryl;

R₅ is alkyl, alkenyl, alkynyl, cycloalkyl, heterocycloalkyl, haloalkyl, haloalkenyl, haloalkynyl, halocycloalkyl, haloheterocycloalkyl, —CN, —NO₂, —OR₃, —SR₃, —N(R₃)₂, —C(O)R₃, —C(O)N(R₃)₂, —NR₃C(O)R₃, —S(O)₂N(R₃)₂, —NR₃S(O)₂R₃, alkyl substituted with 1-4 substituent(s) independently selected from F, Cl, Br, I, or R₆, cycloalkyl substituted with 1-4 substituent(s) independently selected from F, Cl, Br, I, or R₆, or heterocycloalkyl substituted with 1-4 substituent(s) independently selected from F, Cl, Br, I, or R₆;

R₆ is —CF₃, —CN, —NO₂, —OR₃, —SR₃, —N(R₃)₂, —C(O)R₃, —C(O)N(R₃)₂, —NR₃C(O)R₃, —S(O)₂N(R₃)₂, or —NR₃S(O)₂R₃;

R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

Each R₉ is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;

or pharmaceutical composition, pharmaceutically acceptable salt, racemic mixture, or pure enantiomer thereof useful to treat any one of or combination of cognitive and attention deficit symptoms of Alzheimer's, neurodegeneration associated with diseases such as Alzheimer's disease, pre-senile dementia (mild cognitive impairment), senile dementia, schizophrenia or psychosis and related associated cognitive deficits, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, symptoms associated with pain; pain and inflammation (rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster); cancer (multiple myeloma; acute and chronic myelogenous leukemia; or cancer-associated cachexia); diabetes (pancreatic beta cell destruction; or type I and type II diabetes); wound healing (healing burns, and wounds in general including from surgery); bone fracture healing; ischemic heart disease, or stable angina pectoris.

In another aspect, the invention includes a combination therapy for treating a mammal or preparing a medicament to treat a mammal as discussed herein. The compounds of Formula I and the other drug(s)/agent(s) can be administered simultaneously or at separate intervals. When administered simultaneously the compounds of Formula I and the other drug(s)/agent(s) can be incorporated into a single pharmaceutical composition. Alternatively, separate compositions, i.e., one containing compounds of Formula I and one or more containing the other drug(s), can be administered during a therapeutic interval.

A positive allosteric modulator of α7 nAChR will effectively activate the endogenous α7 nAChR if there is sufficient agonist in the brain to at least partially stimulate this receptor. Therefore, a positive allosteric modulator of α7 nAChR can be administered alone to treat the disease or conditions discussed herein. In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. In such cases, the positive allosteric modulator of α7 nAChR is administered in combination with another agent that affects the level of agonist.

The present invention includes the intermediates, the processes to make them and the compounds of the present invention and salts thereof, pharmaceutical compositions containing the active compounds of the present invention, and methods to treat the identified diseases.

The compounds of Formula I exist in tautomeric or enantiomeric forms, all of which are included within the scope of the invention. The various optical isomers may be isolated by separation of a racemic mixture of the compounds using conventional techniques, e.g., fractional crystallization, or chiral HPLC. Alternatively, the individual enantiomers may be made by reaction of the appropriate optically active starting materials under reaction conditions which will not cause racemization.

Abbreviations which are well known to one of ordinary skill in the art may be used (e.g., “Ph” for phenyl, “Me” for methyl, “Et” for ethyl, “h” or “hr” or “hrs” for hour or hours, “min” for minute or minutes, and “rt” for room temperature).

All temperatures are in degrees Centigrade.

Room temperature is within the range of 15-25 degrees Celsius.

Pre-senile dementia is also known as mild cognitive impairment.

ACh refers to acetylcholine.

AChR refers to acetylcholine receptor.

nAChR refers to nicotinic acetylcholine receptor.

mAChR refers to muscarinic acetylcholine receptor.

PAM refers to positive allosteric modulator.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

MLA refers to methyllycaconitine.

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to diisopropylethylamine.

DMAP refers to 4-(dimethylamino)pyridine.

BINAP refers to racemic-2,2′-bis(diphenylphosphino)-1,1′-binaphthyl.

Pd₂(dba) refers to tris(dibenzylideneacetone)dipalladium (0).

Ether refers to diethyl ether.

Na₂SO₄ refers to sodium sulfate.

K₂CO₃ refers to potassium carbonate.

MgSO₄ refers to magnesium sulfate.

When Na₂SO₄, K₂CO₃, or MgSO₄ is used as a drying agent, it is anhydrous.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Mammal denotes human and other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

Parr refers to the name of the company who sells the jars used for conducting reactions under pressure.

PSI means pound per square inch.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

Non-inclusive examples of heterocycloalkyl include, but are not limited to, tetrahydrofurano, tetrahydropyrano, pyrrolidino, piperidino, piperazino, morpholino, thiomorpholino, pyrazolo, 1,1-dioxidothiomorpholino, azetidino, azetidinono, oxindolo, dihydroimidazolo, and pyrrolidinono.

Compounds of the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired effect. As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

The amount of therapeutically effective compound(s) that is administered and the dosage regimen for treating a disease condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of Formula I. The pharmaceutical compositions may contain active ingredient in the range of about 0.001 to 100 mg/kg/day for an adult, preferably in the range of about 0.01 to about 50 mg/kg/day for an adult. A total daily dose of about 1 to 1000 mg of active ingredient may be appropriate for an adult. The daily dose can be administered in one to four doses per day.

In addition to the compound(s) of Formula I, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art.

Compounds of the present invention can enhance the efficacy of agonists at nicotinic receptors, and, are, therefore, referred to as “positive allosteric modulators.” Cholinergic receptors normally bind the endogenous neurotransmitter ACh. AChRs in the mammalian central nervous system can be divided into mAChR and nAChR subtypes based on the agonist activities of muscarine and nicotine, respectively. The nAChRs are ligand-gated ion channels containing five subunits. Members of the nAChR gene family have been divided into two groups based on their sequences: α and β. Three of the α subunits (α7, α8, and α9) form functional receptors when expressed alone and presumably form homooligomeric receptors.

α7 nAChR is a ligand-gated Ca⁺⁺ channel formed by a homopentamer of α7 subunits. Previous studies have established that in the central nervous system α-btx binds selectively to this homopetameric, α7 nAChR subtype, and that α7 nAChR has a high affinity binding site for both α-btx and MLA. α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a sequence homology with α7 nAChR but functionally the two ligand-gated ion channels are very different. For example, α7 nAChR is rapidly desensitized, is highly permeable to calcium and is activated by acetylcholine and nicotine. 5HT₃R is desensitized slowly, is relatively impermeable to calcium and is activated by serotonin. The pharmacology of the α7 nAChR and 5HT₃R channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R.

An allosteric transition state model of the nAChR involves at least a resting state (closed), an activated state (open), and a “desensitized” closed channel state (Changeux, J. and Edelstein, S. J., Curr. Opin. Neurobiolo. 2001 11(3): 369-77; Itier, V. and Bertrand, D., FEBS Lett 2001, 504(3): 118-25). Different nAChR ligands can, therefore, differentially stabilize the conformational state to which they preferentially bind. For example, the agonists ACh and (−)-nicotine drive the nAChR to a desensitized state.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and attention-deficit disorders (Potter, A. et. al., Psychopharmacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

However, treatment with nicotinic receptor agonists which act at the same site as ACh is problematic because ACh not only activates, but also blocks receptor activity through processes which include desensitization and uncompetitive blockade (open-channel block). Forman & Miller (1988) Biophysical J. 54(1): 149-158. Furthermore, prolonged activation may up regulate receptor expression and induce a long-lasting inactivation (Olale, F., et al., J. Pharmacol. Exp. Ther. 1997, 283(2):675-83; Kuryatov, A. et al., Eur. J. Pharmacol. 2000, 393(1-3):11-21; Kawai, H. and Berg, D. K., J. Neurochem. 2001, 78(6):1367-78; Buisson, B. and Bertrand, D., J. Neurosci. 2001, 21(6):1819-29). Therefore, agonists of nAChRs can be expected to reduce activity as well as enhance it. At nicotinic receptors in general, and, of particular note, at the α7-nicotinic receptor, desensitization limits the duration that the channel remains in the active state during agonist application.

The present invention provides a means to increase α7 nAChR function in the brain and other organs, tissues and cells of the body by making these receptors more sensitive to activation by an agonist, including, but not limited to, ACh which is the endogenous agonist. Galantamine, an alkaloid originally obtained from bulbs of snowdrops, is a weak cholinesterase inhibitor and is reported to be a positive allosteric modulator of some nicotinic receptors (Santos, M. D., et al, Mol. Pharmacol. 2002, 61(5):1222-1234). The advantage of this invention is that a drug that works as a PAM of the α7 nAChR will provide long-lasting therapeutic value and will have a minimal risk of loss of therapeutic efficacy because of receptor desensitization. A PAM will also be a relatively safe drug because it acts to amplify the actions of an endogenous neurotransmitter.

Schizophrenia is a complex multifactorial illness caused by genetic and non-genetic risk factors that produce a constellation of positive and negative symptoms. The positive symptoms include delusions and hallucinations and the negative symptoms include deficits in affect, attention, cognition and information processing. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating both the positive and some of the negative symptoms of this disease. Clozapine's utility as a drug is greatly limited because continued use leads to an increased risk of agranulocytosis and seizure. No other antipsychotic drug is effective in treating the negative symptoms of schizophrenia. This is significant because the restoration of cognitive functioning is the best predictor of a successful clinical and functional outcome of schizophrenic patients (Green, M. F., Am J Psychiatry, 153:321-30, 1996). By extension, it is clear that better drugs are needed to treat the cognitive disorders of schizophrenia in order to restore a better state of mental health to patients with this disorder.

One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

A positive allosteric modulator of α7 nAChR will effectively activate the endogenous α7 nAChR if there is sufficient agonist in the brain to at least partially stimulate this receptor. Therefore, a positive allosteric modulator of α7 nAChR can be administered alone to treat the disease or conditions discussed herein.

In certain diseases, however, it is possible that the full therapeutic efficacy of a positive allosteric modulator of α7 nAChR will be limited by suboptimal levels of agonist which in turn leads to a suboptimal activation of the endogenous α7 nAChR in the presence of a positive allosteric modulator. For example but not limitation, it is well established that in Alzheimer's disease, there is a loss of ACh from the brains of the patients with this disease and this loss is correlated with disease progression. In this case, the primary role of combination therapy is to treat patients with therapeutic agents that directly activate the endogenous of α7 nAChR in combination with a positive allosteric modulator of α7 nAChR to achieve maximal efficacy. Thus, in Alzheimer's disease, it is likely that the full therapeutic efficacy of a positive allosteric modulator of α7 nAChR could be enhanced if combination therapy is used. This combination therapy applies to other diseases or conditions discussed herein where there is a loss of ACh. One of ordinary skill in the art would recognize for which disease or conditions this combination therapy would be useful.

The compounds of the present invention are α7 nAChR PAMs and may be used to treat a wide variety of diseases. For example, they may be used in treating schizophrenia, or psychosis.

Schizophrenia is a disease having multiple aspects. Currently available drugs are generally aimed at controlling the positive aspects of schizophrenia, such as delusions. One drug, Clozapine, is aimed at a broader spectrum of symptoms associated with schizophrenia. This drug has many side effects and is thus not suitable for many patients. Thus, there is a need for a drug to treat the cognitive and attention deficits associated with schizophrenia. Similarly, there is a need for a drug to treat the cognitive and attention deficits associated with schizoaffective disorders, or similar symptoms found in the relatives of schizophrenic patients.

Psychosis is a mental disorder characterized by gross impairment in the patient's perception of reality. The patient may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

There are a variety of antipsychotic drugs. The conventional antipsychotic drugs include Chlorpromazine, Fluphenazine, Haloperidol, Loxapine, Mesoridazine, Molindone, Perphenazine, Pimozide, Thioridazine, Thiothixene, and Trifluoperazine. These drugs all have an affinity for the dopamine 2 receptor.

These conventional antipsychotic drugs have several side effects, including sedation, weight gain, tremors, elevated prolactin levels, akathisia (motor restlessness), dystonia and muscle stiffness. These drugs may also cause tardive dyskinesia. Unfortunately, only about 70% of patients with schizophrenia respond to conventional antipsychotic drugs. For these patients, atypical antipsychotic drugs are available.

Atypical antipsychotic drugs generally are able to alleviate positive symptoms of psychosis while also improving negative symptoms of the psychosis to a greater degree than conventional antipsychotics. These drugs may improve neurocognitive deficits. Extrapyramidal (motor) side effects are not as likely to occur with the atypical antipsychotic drugs, and thus, these atypical antipsychotic drugs have a lower risk of producing tardive dyskinesia. Finally these atypical antipsychotic drugs cause little or no elevation of prolactin. Unfortunately, these drugs are not free of side effects. Although these drugs each produce different side effects, as a group the side effects include: agranulocytosis; increased risk of seizures, weight gain, somnolence, dizziness, tachycardia, decreased ejaculatory volume, and mild prolongation of QTc interval.

In a combination therapy to treat multiple symptoms of diseases such as schizophrenia, the compounds of Formula I and the anti-psychotic drugs can be administered simultaneously or at separate intervals. When administered simultaneously the compounds of Formula I and the anti-psychotic drugs can be incorporated into a single pharmaceutical composition, e.g., a pharmaceutical combination therapy composition. Alternatively, two separate compositions, i.e., one containing compounds of Formula I and the other containing anti-psychotic drugs, can be administered simultaneously. Examples of anti-psychotic drugs, in addition to those listed above, include, but are not limited to, Thorazine, Mellaril, Trilafon, Navane, Stelazine, Permitil, Prolixin, Risperdal, Zyprexa, Seroquel, ZELDOX, Acetophenazine, Carphenazine, Chlorprothixene, Droperidol, Loxapine, Mesoridazine, Molindone, Ondansetron, Pimozide, Prochlorperazine, and Promazine.

A pharmaceutical combination therapy composition can include therapeutically effective amounts of the compounds of Formula I, noted above, and a therapeutically effective amount of anti-psychotic drugs. These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and maybe formulated as sustained relief dosage forms and the like.

When separately administered, therapeutically effective amounts of compositions containing compounds of Formula I and anti-psychotic drugs are administered on a different schedule. One may be administered before the other as long as the time between the two administrations falls within a therapeutically effective interval. A therapeutically effective interval is a period of time beginning when one of either (a) the compounds of Formula I, or (b) the anti-psychotic drugs is administered to a human and ending at the limit of the beneficial effect in the treatment of schizophrenia or psychosis of the combination of (a) and (b). The methods of administration of the compounds of Formula I and the anti-psychotic drugs may vary. Thus, either agent or both agents may be administered rectally, topically, orally, sublingually, or parenterally.

As discussed, the compounds of the present invention are α7 nAChR PAMs. Therefore, as another aspect of the present invention, the compounds of the present invention may be used to treat a variety of diseases including cognitive and attention deficit symptoms of Alzheimer's, neurodegeneration associated with diseases such as Alzheimer's disease, pre-senile dementia (also known as mild cognitive impairment), and senile dementia.

Alzheimer's disease has many aspects, including cognitive and attention deficits. Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for a drug that stimulates a portion of the cholinergic pathways and thereby provides improvement in the cognitive and attention deficits associated with Alzheimer's disease without the side effects created by nonspecific stimulation of the cholinergic pathways.

Neurodegeneration is a common problem associated with diseases such as Alzheimer's disease. While the current drugs treat some of the symptoms of this disease, they do not control the underlying pathology of the disease. Accordingly, it would be desirable to provide a drug that can slow the progress of Alzheimer's disease.

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

As discussed, the compounds of the present invention are α7 nAChR PAMs. Therefore, other diseases to be treated with compounds of the present invention include treating the cognitive and attention deficits as well as the neurodegeneration associated with attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, or symptoms associated with pain.

Attention deficit disorder is generally treated with methylphenidate, an amphetamine-like molecule that has some potential for abuse. Accordingly, it would be desirable to provide a drug that treats attention deficit disorder while having fewer side effects than the currently used drug.

Attention deficit hyperactivity disorder, otherwise known as ADHD, is a neurobehavioral disorder affecting 3-5% of all American children. ADHD concerns cognitive alone or both cognitive and behavioral actions by interfering with a person's ability to stay on a task and to exercise age-appropriate inhibition. Several types of ADHD exist: a predominantly inattentive subtype, a predominantly hyperactive-impulsive subtype, and a combined subtype. Treatment may include medications such as methylphenidate, dextroamphetamine, or pemoline, which act to decrease impulsivity and hyperactivity and to increase attention. No “cure” for ADHD currently exists. Children with the disorder seldom outgrow it; therefore, there is a need for appropriate medicaments.

The compounds of the present invention can also be combined with a psychostimulant or a monoamine reuptake inhibitor and optionally combined with an alpha7 nAChR agonist, especially when endogenous agonist is suboptimal.

By combination is meant the administration of the two agents within a month or two or less of each other, preferably within a week and more preferably at about the same time or within a day or two or less of each other.

In a combination therapy to treat ADHD, the compounds of Formula I and the psychostimulant or inhibitor can be administered simultaneously or at separate intervals. When administered simultaneously the compounds of Formula I and the psychostimulants or monoamine reuptake inhibitors can be incorporated into a single pharmaceutical composition, e.g., a pharmaceutical combination therapy composition. Alternatively, two separate compositions, i.e., one containing compounds of Formula I and the other containing the psychostimulants or monoamine reuptake inhibitors.

A pharmaceutical combination therapy composition can include therapeutically effective amounts of the compounds of Formula I, noted herein, and a therapeutically effective amount of the psychostimulants or monoamine reuptake inhibitors. While psychostimulants and monoamine reuptake inhibitors control the activity level, and attention, they are not effective in treating the co-morbid or concomitant deficit in cognitive that is associated with ADHD. The combination therapy will be more effective at treating this disease because a PAM and optionally an α7 nAChR agonist will treat the underlying cognitive dysfunction in the disorder and the other two classes of drugs will treat the behavioral problems associated with ADHD. The combined administration of the compounds of Formula I and optionally an agonist and the psychostimulant or monoamine reuptake inhibitor is expected to require less of the generally-prescribed dose for either agent when used alone and or is expected to result in less frequent administration of either or both agents. The skilled clinician may in fact learn that behavioral problems are secondary to the cognitive problems and can be treated with lower dosages of the inhibitors. Determining such dosages should be a routine determination by one skilled in the art of treating patients with ADHD.

Mood and affective disorders fall within a large group of diseases, including monopolar depression and bi-polar mood disorder. These diseases are treated with three major classes of compounds. The first group is the heterocyclic antidepressant (HCA's). This group includes the well-known tricyclic antidepressants. The second group of compounds used to treat mood disorders is the monoamine oxidase inhibitors (MAOI's) that are used in particular types of diseases. The third drug is lithium. Common side effects from HCA's are sedation and weight gain. In elderly patients with organic brain disease, the side effects of HCA's can also include seizures and behavioral symptoms. The main side effects from using MAOI's occur from dietary and drug interactions. Benign side effects from the use of lithium include, but are not limited to, weight gain, nausea, diarrhea, polyuria, polydipsia, and tremor. Toxic side effects from lithium can include persistent headache, mental confusion, and may reach seizures and cardiac arrhythmias. Therefore, agents with less side effects or interactions with food or other medications would be useful.

Depression is a mood disorder of varying lengths of normally several months to more than two years and of varying degrees of feelings involving sadness, despair, and discouragement. The heterocyclic antidepressants (HCA's) are currently the largest class of antidepressants, but monoamine oxidase inhibitors (MAOI's) are used in particular types of depression. Common side effects from HCA's are sedation and weight gain. In elderly patients with organic brain disease, the side effects from HCA's can also include seizures and behavioral symptoms. The main side effects from using MAOI's occur from dietary and drug interactions. Therefore, agents with fewer side effects would be useful.

Borderline personality disorder, although not as well known as bipolar disorder, is more common. People having borderline personality disorder suffer from a disorder of emotion regulation. Pharmaceutical agents are used to treat specific symptoms, such as depression or thinking distortions.

Acquired immune deficiency syndrome (AIDS) results from an infection with the human immunodeficiency virus (HIV). This virus attacks selected cells and impairs the proper function of the immune, nervous, and other systems. HIV infection can cause other problems such as, but not limited to, difficulties in thinking, otherwise known as AIDS dementia complex. Therefore, there is a need to drugs to relieve the confusion and mental decline of persons with AIDS.

Amyotrophic lateral sclerosis, also known as Lou Gehrig's disease, belongs to a class of disorders known as motor neuron diseases wherein specific nerve cells in the brain and spinal cord gradually degenerate to negatively affect the control of voluntary movement. Currently, there is no cure for amyotrophic lateral sclerosis although patients may receive treatment from some of their symptoms and although Riluzole has been shown to prolong the survival of patients. Therefore, there is a need for a pharmaceutical agent to treat this disease.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

General anxiety disorder (GAD) occurs when a person worries about things such as family, health, or work when there is no reason to worry and is unable not to worry. About 3 to 4% of the U.S. population has GAD during the course of a year. GAD most often strikes people in childhood or adolescence, but can begin in adulthood, too. It affects women more often than men. Currently, treatment involves cognitive-behavioral therapy, relaxation techniques, and biofeedback to control muscle tension and medications such as benzodiazepines, imipramine, and buspirone. These drugs are effective but all have side-effect liabilities. Therefore, there is a need of a pharmaceutical agent to address the symptoms with fewer side effects.

Dementia with Lewy Bodies is a neurodegenerative disorder involving abnormal structures known as Lewy bodies found in certain areas of the brain. Symptoms of dementia with Lewy bodies include, but are not limited to, fluctuating cognitive impairment with episodic delirium. Currently, treatment concerns addressing the parkinsonian and psychiatric symptoms. However, medicine to control tremors or loss of muscle movement may actually accentuate the underlying disease of dementia with Lewy bodies. Therefore, there is a need of a pharmaceutical agent to treat dementia with Lewy bodies.

Age-related macular degeneration (AMD) is a common eye disease of the macula which is a tiny area in the retina that helps produce sharp, central vision required for “straight ahead” activities that include reading and driving. Persons with AMD lose their clear, central vision. AMD takes two forms: wet and dry. In dry AMD, there is a slow breakdown of light-sensing cells in the macula. There currently is no cure for dry AMD. In wet AMD, new, fragile blood vessels growing beneath the macula as dry AMD worsens and these vessels often leak blood and fluid to cause rapid damage to the macula quickly leading to the loss of central vision. Laser surgery can treat some cases of wet AMD. Therefore, there is a need of a pharmaceutical agent to address AMD.

Parkinson's disease is a neurological disorder characterized by tremor, hypokinesia, and muscular rigidity. Currently, there is no treatment to stop the progression of the disease. Therefore, there is a need of a pharmaceutical agent to address Parkinson's.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of tardive dyskinesia.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions or hallucinations. Therefore, there is a need for a pharmaceutical agent to treat the progressive deterioration of social skills and changes in personality and to address the symptoms with fewer side effects.

Post-traumatic stress disorder (PTSD) is a form of anxiety triggered by memories of a traumatic event that directly affected the patient or that the patient may have witnessed. The disorder-commonly affects survivors of traumatic events including sexual assault, physical assault, war, torture, natural disasters, an automobile accident, an airplane crash, a hostage situation, or a death camp. The affliction also can affect rescue workers at an airplane crash or a mass shooting, someone who witnessed a tragic accident or someone who has unexpectedly lost a loved one. Treatment for PTSD includes cognitive-behavioral therapy, group psychotherapy, and medications such as Clonazepam, Lorazepam and selective serotonin-reuptake inhibitors such as Fluoxetine, Sertraline, Paroxetine, Citalopram and Fluvoxamine. These medications help control anxiety as well as depression. Various forms of exposure therapy (such as systemic desensitization and imaginal flooding) have all been used with PTSD patients. Exposure treatment for PTSD involves repeated reliving of the trauma, under controlled conditions, with the aim of facilitating the processing of the trauma. Therefore, there is a need for better pharmaceutical agents to treat Post traumatic stress disorder.

Dysregulation of food intake associated with eating disease, including bulemia nervosa and anorexia nervosa, involve neurophysiological pathways. Anorexia nervosa is hard to treat due to patients not entering or remaining in after entering programs. Currently, there is no effective treatment for persons suffering from severe anorexia nervosa. Cognitive behavioral therapy has helped patients suffering from bulemia nervosa; however, the response rate is only about 50% and current treatment does not adequately address emotional regulation. Therefore, there is a need for pharmaceutical agents to address neurophysiological problems underlying diseases of dysregulation of food intake.

Cigarette smoking has been recognized as a major public health problem for a long time. However, in spite of the public awareness of health hazard, the smoking habit remains extraordinarily persistent and difficult to break. There are many treatment methods available, and yet people continue to smoke. Administration of nicotine transdermally, or in a chewing gum base is common treatments. However, nicotine has a large number of actions in the body, and thus can have many side effects. It is clear that there is both a need and a demand of long standing for a convenient and relatively easy method for aiding smokers in reducing or eliminating cigarette consumption. A drug that could selectively stimulate only certain of the nicotinic receptors would be useful in smoke cessation programs.

Smoke cessation programs may involve oral dosing of the drug of choice. The drug may be in the form of tablets. However, it is preferred to administer the daily dose over the waking hours, by administration of a series of incremental doses during the day. The preferred method of such administration is a slowly dissolving lozenge, troche, or chewing gum, in which the drug is dispersed. Another drug in treating nicotine addiction is Zyban. This is not a nicotine replacement, as are the gum and patch. Rather, this works on other areas of the brain, and its effectiveness is to help control nicotine craving or thoughts about cigarette use in people trying to quit. Zyban is not very effective and effective drugs are needed to assist smokers in their desire to stop smoking. These drugs may be administered transdermally through the use of skin patches. In certain cases, the drugs may be administered by subcutaneous injection, especially if sustained release formulations are used.

Drug use and dependence is a complex phenomenon, which cannot be encapsulated within a single definition. Different drugs have different effects, and therefore different types of dependence. Drug dependence has two basic causes, that is, tolerance and physical dependence. Tolerance exists when the user must take progressively larger doses to produce the effect originally achieved with smaller doses. Physical dependence exists when the user has developed a state of physiologic adaptation to a drug, and there is a withdrawal (abstinence) syndrome when the drug is no longer taken. A withdrawal syndrome can occur either when the drug is discontinued or when an antagonist displaces the drug from its binding site on cell receptors, thereby counteracting its effect. Drug dependence does not always require physical dependence.

In addition drug dependence often involves psychological dependence, that is, a feeling of pleasure or satisfaction when taking the drug. These feelings lead the user to repeat the drug experience or to avoid the displeasure of being deprived of the drug. Drugs that produce strong physical dependence, such as nicotine, heroin and alcohol are often abused, and the pattern of dependence is difficult to break. Drugs that produce dependence act on the CNS and generally reduce anxiety and tension; produce elation, euphoria, or other pleasurable mood changes; provide the user feelings of increased mental and physical ability; or alter sensory perception in some pleasurable manner. Among the drugs that are commonly abused are ethyl alcohol, opioids, anxiolytics, hypnotics, cannabis (marijuana), cocaine, amphetamines, and hallucinogens. The current treatment for drug-addicted people often involves a combination of behavioral therapies and medications. Medications, such as methadone or LAAM (levo-alpha-acetyl-methadol), are effective in suppressing the withdrawal symptoms and drug craving associated with narcotic addiction, thus reducing illicit drug use and improving the chances of the individual remaining in treatment. The primary medically assisted withdrawal method for narcotic addiction is to switch the patient to a comparable drug that produces milder withdrawal symptoms, and then gradually taper off the substitute medication. The medication used most often is methadone, taken orally once a day. Patients are started on the lowest dose that prevents the more severe signs of withdrawal and then the dose is gradually reduced. Substitutes can be used also for withdrawal from sedatives. Patients can be switched to long-acting sedatives, such as diazepam or phenobarbital, which are then gradually reduced.

Gilles de la Tourette's Syndrome is an inherited neurological disorder. The disorder is characterized by uncontrollable vocal sounds called tics and involuntary movements. The symptoms generally manifest in an individual before the person is 18 years of age. The movement disorder may begin with simple tics that progress to multiple complex tics, including respiratory and vocal ones. Vocal tics may begin as grunting or barking noises and evolve into compulsive utterances. Coprolalia (involuntary scatologic utterances) occurs in 50% of patients. Severe tics and coprolalia may be physically and socially disabling. Tics tend to be more complex than myoclonus, but less flowing than choreic movements, from which they must be differentiated. The patient may voluntarily suppress them for seconds or minutes.

Currently simple tics are often treated with benzodiazepines. For simple and complex tics, Clonidine may be used. Long-term use of Clonidine does not cause tardive dyskinesia; its limiting adverse effect is hypotension. In more severe cases, antipsychotics, such as Haloperidol may be required, but side effects of dysphoria, parkinsonism, akathisia, and tardive dyskinesia may limit use of such antipsychotics. There is a need for safe and effective methods for treating this syndrome.

Glaucoma is within a group of diseases occurs from an increase in intraocular pressure causing pathological changes in the optical disk and negatively affects the field of vision. Medicaments to treat glaucoma either decrease the amount of fluid entering the eye or increase drainage of fluids from the eye in order to decrease intraocular pressure. However, current drugs have drawbacks such as not working over time or causing side effects so the eye-care professional has to either prescribe other drugs or modify the prescription of the drug being used. There is a need for safe and effective methods for treating problems manifesting into glaucoma.

Ischemic periods in glaucoma cause release of excitotoxic amino acids and stimulate inducible form of nitric oxide synthase (iNOS) leading to neurodegeneration. A PAM stimulates an agonist to affect the release of inhibitory amino acids such as GABA which will dampen hyperexcitablity. PAMs are also directly neuroprotective on neuronal cell bodies. Thus, PAMs have the potential to be neuroprotective in glaucoma.

Persons afflicted with pain often have what is referred to as the “terrible triad” of suffering from the pain, resulting in sleeplessness and sadness, all of which are hard on the afflicted individual and that individual's family. Pain can manifest itself in various forms, including, but not limited to, headaches of all severity, back pain, neurogenic, and pain from other ailments such as arthritis and cancer from its existence or from therapy to irradicate it. Pain can be either chronic (persistent pain for months or years) or acute (short-lived, immediate pain to inform the person of possible injury and need of treatment). Persons suffering from pain respond differently to individual therapies with varying degrees of success. There is a need for safe and effective methods for treating pain.

TNF-α is a pro-inflammatory cytokine secreted by a variety of cells, including monocytes and macrophages, in response to many inflammatory stimuli (e.g., lipopolysaccharide—LPS) or external cellular stresses (e.g., osmotic shock and peroxide). Elevated levels of TNF-α over basal levels have been implicated in mediating or exacerbating a number of diseases or conditions involving inflammation, pain, cancer, and diabetes. TNF-α is upstream in the cytokine cascade of inflammation. By decreasing levels of TNF-α, not only are levels of TNF-α minimized, but also elevated levels of other inflammatory and proinflammatory cytokines, such as IL-1, IL-6, and IL-8. TNF-α plays a role in head trauma, stroke, and ischemia. Shohami et al., J. Cereb. Blood Flow Metab., 14, 615 (1994). TNF-α promotes the infiltration of other cytokines (IL-1beta, IL-6) and also chemokines, which promote neutrophil infiltration into the infarct area. TNF-α plays a role in promoting certain viral life cycles and disease states associated with them; for instance, TNF-α secreted by monocytes induced elevated levels of HIV expression in a chronically infected T cell clone. Clouse et al., J. Immunol., 142, 431 (1989); Lahdevirte et al., Am. J. Med. 85, 289 (1988). TNF-α is associated with the HIV mediated states of cachexia due to cancer and muscle degradation.

TNF-α plays a role in pancreatic beta cell destruction and diabetes. Yoon J W, and Jun H S, Diabetologia, 44(3), 271-285 (2001). Pancreatic beta cells produce insulin which helps mediate blood-glucose homeostasis. Deterioration of pancreatic beta cells often accompanies type I diabetes. Pancreatic beta cell functional abnormalities may occur in patients with type II diabetes. Type II diabetes is characterized by a functional resistance to insulin. Further, type II diabetes is also often accompanied by elevated levels of plasma glucagon and increased rates of hepatic glucose production.

In rheumatoid arthritis, TNF-α induces synoviocytes and chondrocytes to produce collagenase and neutral proteases, which lead to tissue destruction within the arthritic joints. In a model of arthritis (collagen-induced arthritis (CIA) in rats and mice), intra-articular administration of TNF-α either prior to or after the induction of CIA led to an accelerated onset of arthritis and a more severe course of the disease. Brahn et al., Lymphokine Cytokine Res., 11, 253 (1992); and Cooper, Clin. Exp. Immunol., 898, 244 (1992). By reducing TNF-α levels, the resulting levels of synoviocytes and chondrocytes are also reduced to prevent or minimize the effects of rheumatoid arthritis.

The compounds of the present invention are useful to treat, or used to prepare a medicament used to treat, diseases or conditions where a mammal receives symptomatic relief from the decrease of levels of TNF-α; these diseases or conditions include, but are not limited to, any one or more or combination of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, or HIV-3; CMV; influenza, adenovirus, a herpes virus (including HSV-1, HSV-2); herpes zoster; multiple myeloma; acute and chronic myelogenous leukemia; cancer-associated cachexia; pancreatic beta cell destruction; type I or type II diabetes.

Some nicotinic receptors regulate vascular angiogenesis; for example, the binding of nicotine to the alpha-7 nAChR stimulates DNA synthesis and proliferation of vascular endothelial cells. Villablanca, supra. The compounds of the present invention are also useful to treat, or are used to prepare a medicament to treat, diseases or conditions where a mammal receives symptomatic relief from the stimulation of vascular angiogenesis; these diseases or conditions include, but not limited to, any one or more of the following: wound healing (healing burns, and wounds in general including from surgery), bone fracture healing, ischemic heart disease, and stable angina pectoris.

Compounds of Formula I can be prepared as shown in Scheme 1. The syntheses shown in the following schemes use intermediates where W^{A-1}, W^{A-2}, W^{A-3}, W^{A-4}, and W^{A-5} for the final compounds would be CR_{A}. One of ordinary skill in the art could make the corresponding compounds where up to four of W^{A-1}, W^{A-2}, W^{A-3}, W^{A-4}, and W^{A-5} are N making non-critical changes to the methods discussed. The intermediates leading to the B moiety of Formula I can also be prepared by one of ordinary skill in the art with the methods discussed herein or using known procedures or commercially available intermediates. The following discussion is not intended to limit the scope of the invention but is for exemplification only. Methods to synthesize ureas and thioureas of Formula I are well known to one skilled in the art. For example, aryl isocyanates or aryl isothiocyanates (II) or heteroaryl isocyanates or heteroaryl isothiocyanates (I) can be reacted with aminoheterocycles or anilines to provide the desired urea or thiourea using procedures described in J. Med. Chem. 1996, 39, 304; J. Med. Chem. 1999, 39, 4382; Pharmazie 1999, 54, 19; J. Chem. Soc. 1963, 40, 369; J. Chem. Soc. Perkin Trans. I 1977, 1616; and Synth. Commun. 2001, 31, 781. Alternatively, compounds of formula IV or V can be reacted with an aminoheterocycle or an aniline to provide the desired urea or thiourea using procedures described in J. Med. Chem. 1999, 39, 304; J. Med. Chem. (1995) 38, 855.

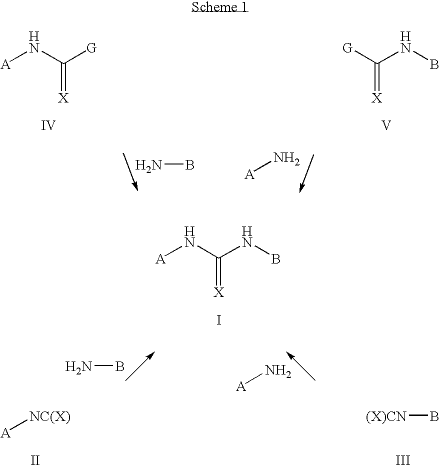

where G is 4-nitro-phenoxy, phenoxy, or imidazol-1-yl.

Compounds of Formula III can be prepared as shown in Scheme 2. Methods to synthesize isocyanates or isothiocyanates of Formula III are well known to one skilled in the art. For example, an aminoheterocycle can be reacted with excess phosgene (or phosgene equivalent) or thiophosgene in refluxing ethyl acetate to provide the heterocyclic isocyanate as described in U.S. Pat. No. 3,759,940. Alternatively, heterocyclic isocyanates III can be prepared from the corresponding carboxylic acid or acid derivative by treatment with an azide source such as sodium azide or diphenylphosphoryl azide (DPPA) followed by a Curtius-type rearrangement using procedures described in J. Org. Chem. 1985, 50, 5723; J. Org. Chem. 1997, 62, 3013. Compounds of Formula V can be synthesized using procedures well known to one skilled in the art (see DE 1816696; and Greene, T. W. and Wuts, P. G. M. “Protective Groups in Organic Synthesis”, 3rd Edition, p. 549, New York:Wiley, (1999)). The requisite aminoheterocycles or heterocyclic carboxylic acids can be obtained from commercial sources or can be synthesized by known procedures.

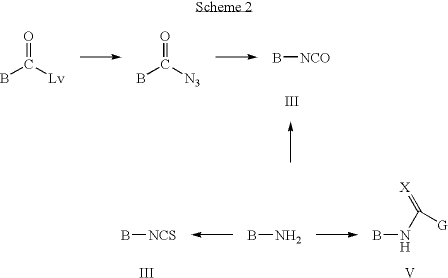

where G is as defined for Scheme 1 and Lv is OH, Cl, or —NH—NH₂.

It will be apparent to those skilled in the art that the aryl isocyanates or aryl isothiocyanates II can be obtained commercially or can be synthesized by known procedures. Compounds of Formula II can be prepared in a manner exactly analogous to the procedures used for the preparation of compounds of Formula III. The requisite substituted anilines can be purchased from commercial sources or prepared using procedures outlined in J. Org. Chem. 1997, 62, 6471. Alternatively, aryl isocyanates II can be prepared from the corresponding carboxylic acid or acid derivative by treatment with an azide source such as sodium azide or diphenylphosphoryl azide (DPPA) followed by a Curtius-type rearrangement using procedures described in Synth. Commun. 1993, 23, 335; or Heterocycles 1993, 36, 1305. Aryl isothiocyanates II can be prepared according to procedures in J. Org. Chem. 2000, 65, 6237.

Heteroaryl amine linked compounds can be prepared via the general route outlined in Scheme 3. A substituted 2-bromo-nitrobenzene is treated with sodium alkoxideoxide to give the O-substituted compound. This is coupled with requisite aminopyridine via a palladium catalysis (see, Yang, B. H. and Buchwald, S. L. Journal of Organometallic Chem, 1999, 576, 125.146.) The nitro group is reduced to its corresponding amine utilizing methods apparent to those skilled in the art and then reacted with either an aryl carbamate or isocyanate as outlined in previous schemes.

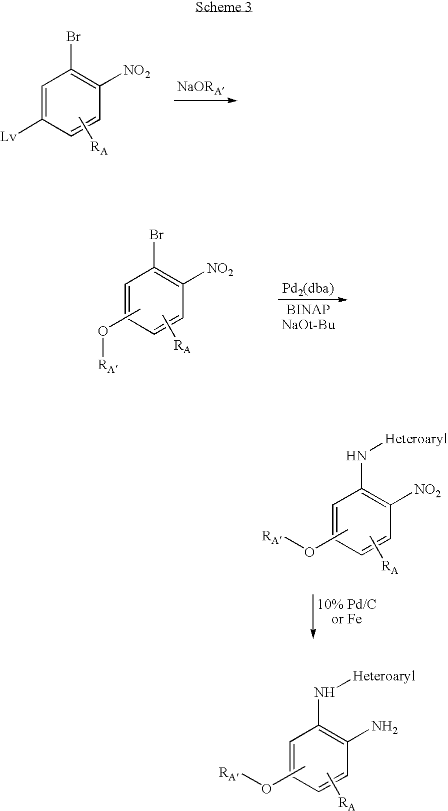

where Lv is F, Cl, Br, SO₂Me.

Heteroaryl linked compounds are prepared via the general route outlined in Scheme 4. A substituted 2-bromo-nitrobenzene is treated with the preformed NaOR to give the alkoxy substituted product, which was reduced to the amine with Fe (powder) or an alternative reduction apparent to one skilled in the art to afford 2-bromo-alkoxy aniline. The Stille coupling of 2-bromo-alkoxy aniline with stannane-heterocycle, which is prepared by the treatment of heterocycle with n-BuLi and tributyltin chloride (Joullie, Tetrahedron Lett. 1994, 35, 7719-22).

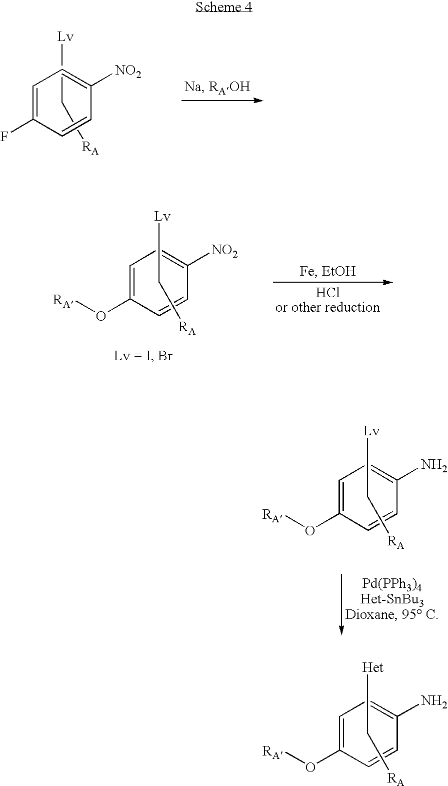

Using the procedures discussed herein and the appropriate starting materials that are either commercially available or readily prepared by one of ordinary skill in the art using known procedures, the compounds of formula I can be prepared where R_{A′} is substituted alkyl and R_{A} is other than H, for example but not limitation, halogen. Furthermore, one of ordinary skill in the art can controlled where the substitution will occur on the phenyl ring of A by selecting the appropriate starting materials as discussed in Schemes 3 and 4.

The following examples are provided as examples and are not intended to limit the scope of this invention to only those provided examples and named compounds.

### EXAMPLE 1

### N-[4-ethoxy-2-(pyridin-4-ylamino)phenyl]-N′-(5-methylisoxazol-3-yl)urea

Absolute EtOH (300 mL) is cooled in an ice bath and sodium (2.1 g) is slowly added. The cooling bath is removed and the resulting mixture allowed to stir at ambient temperature for 2 hours. 2-Bromo-4-fluoro-1-nitrobenzene (6.0 g) is slowly added, and the resulting mixture allowed to stir for 15 hours. A solution of citric acid (1.0 M) is added until the pH was ˜4. Water is added, the volatiles are removed in vacuo and the residue taken up in EtOAc, washed with water, brine, dried (Na₂SO₄) and 2-bromo-4-ethoxy-1-nitrobenzene is crystallized from 1-chlorobutane/hexane. Yield 68%. ¹H NMR (400 MHz, DMSO-d₆) δ 8.04, 7.40, 7.11, 4.15, 1.33.

A mixture of 4-aminopyridine (0.37 g), 2-bromo-4-ethoxy-1-nitrobenzene (1.0 g) Pd₂(bda) (0.15 g), BINAP (0.20 g), and sodium tert-butoxide (0.58 g) is purged with argon, then toluene (40 mL) is added and the resulting mixture heated to 85° C. for 1 hour and then cooled. The solvent is removed in vacuo, and the residue purified using silica gel chromatography (50% to 75% EtOAc/heptane) to give N-(5-ethoxy-2-nitrophenyl)pyridin-4-amine. Yield 84%. MS (ESI+) for C₁₃H₁₃N₃O₃ m/z 260.1 (M−H)⁺.

N-(5-ethoxy-2-nitrophenyl)pyridin-4-amine (0.87 g) is suspended in MeOH (˜200 mL) and 10% Pd/C (0.27 g) is added. The mixture is shaken under 45 psi H₂ for 30 minutes, filtered and concentrated to give 4-ethoxy-N2-pyridin-4-ylbenzene-1,2-diamine as a solid. Yield 89%. ¹H NMR (400 MHz, DMSO-d₆) δ 8.08, 7.98, 6.71, 6.6-6.5, 4.2, 3.87, 1.26.

4-Ethoxy-N²-pyridin-4-ylbenzene-1,2-diamine ((0.33 g), TEA (0.3 mL) and phenyl 5-methylisoxazol-3-ylcarbamate (0.33 g) are dissolved in THF (10 mL). The resulting suspension is heated to 50° C. for 4 hours, and allowed to stir at rt for an additional 12 hours. The solvent is removed in vacuo and Example 1 is obtained as solid crystallized from MeCN. Yield 81%. HRMS (ESI) calcd for C₁₈H₁₉N₅O₃+H 354.1566, found 354.1551.

### EXAMPLE 2

### N-[4-ethoxy-2-(pyridin-3-ylamino)phenyl]-N′-(5-methylisoxazol-3-yl)urea

2-Bromo-4-ethoxy-1-nitrobenzene (1.06 g), 3-aminopyridine (0.38 g), Pd₂(bda) (0.15 g), BINAP (0.20 g), and sodium tert-butoxide (0.59 g) is purged with argon, then toluene (40 mL) is added and the resulting mixture heated to 85° C. for 1 hour and then cooled. The solvent is removed in vacuo, and N-(5-ethoxy-2-nitrophenyl)pyridin-3-amine is purified using silica gel chromatography. Yield 77%. MS (CI+) for C₁₃H₁₃N₃O₃ m/z 260.1 (M+H)⁺.

N-(5-Ethoxy-2-nitrophenyl)pyridin-3-amine (0.79 g) is suspended in MeOH (˜200 mL) and 10% Pd/C is added (0.16 g). The mixture is reacted under 45 psi H₂ for 1 hour, filtered and concentrated to give 4-ethoxy-N²-pyridin-3-ylbenzene-1,2-diamine as a solid. Yield 95%. MS (EI) m/z (rel intensity) 230 (33), 229 (M+, 99), 201 (20), 200 (70), 199 (11), 185 (17), 173 (12), 172 (46), 156 (12), 155 (28).

4-Ethoxy-N²-pyridin-3-ylbenzene-1,2-diamine (0.30 g), TEA (0.28 mL) and phenyl 5-methylisoxazol-3-ylcarbamate (0.32 g) are dissolved in THF (10 mL). The resulting suspension is heated to 50° C. for 4 hours, and allowed to stir at rt for an additional 12 hours. The solvent is removed in vacuo to give Example 2 as a solid crystallized from EtOAc/hexane. Yield 76%. HRMS (ESI) calcd for C₁₈H₁₉N₅O₃+H 354.1566, found 354.1556.

### EXAMPLE 3

### N-[4-ethoxy-2-(pyridin-3-ylamino)phenyl]-N′-[5-(trifluoromethyl)-1,3,4-thiadiazol-2-yl]urea

4-Ethoxy-N²-pyridin-3-ylbenzene-1,2-diamine (0.30 g), DMAP (˜10 mg), 2-isocyanato-5-(trifluoromethyl)-1,3,4-thiadiazole (0.29 g) are suspended in 1:1 THF/DMF (10 mL) and heated to 50° C. for 4 hours, then cooled ambient temperature for an additional 12 hours. The solvents are removed in vacuo and the residue purified by silica gel chromatography (7% [1:9 NH₄OH/MeOH]/CH₂Cl₂ to 10%). Yield 77%. HRMS (ESI) calcd for C₁₇H₁₅N₆O₂SF₃+H 425.1007, found 425.0991.

### EXAMPLE 4

### N-[4-ethoxy-2-(pyridin-2-ylamino)phenyl]-N′-(5-methylisoxazol-3-yl)urea

2-Bromo-4-ethoxy-1-nitrobenzene (1.05 g), 2-aminopyridine (0.39 g) Pd₂(bda) (0.15 g), BINAP (0.20 g), and sodium tert-butoxide (0.59 g) is purged with argon, then toluene (40 mL) is added and the resulting mixture heated to 85° C. for 1 hour and then cooled. The solvent is removed in vacuo, and N-(5-ethoxy-2-nitrophenyl)pyridin-2-amine is purified using silica gel chromatography. Yield 64%. MS (EI) m/z (rel intensity) 259 (M+, 20), 214 (23), 213 (99), 186 (15), 185 (92), 184 (33), 156 (24), 155 (28), 84 (17), 78 (15).

N-(5-ethoxy-2-nitrophenyl)pyridin-2-amine (0.69 g) is suspended in MeOH (˜300 mL) and 10% Pd/C (0.20 g) is added. The mixture is reacted under 45 psi H₂ for 30 minutes. The mixture is filtered and concentrated to give 4-ethoxy-N²-pyridin-2-ylbenzene-1,2-diamine as a solid. Yield quantitative. MS (EI) m/z (rel intensity) 230 (18), 229 (M+, 99), 214 (15), 213 (82), 200 (22), 185 (36), 173 (14), 172 (88), 155 (31), 78 (21).

4-Ethoxy-N²-pyridin-2-ylbenzene-1,2-diamine (0.34 g), TEA (0.34 mL), and phenyl 5-methylisoxazol-3-ylcarbamate (0.36 g) are suspended in THF (10 mL), and heated to 50° C. for 4 hours, then allowed to stir for an additional 12 hours. The solvents are removed in vacuo and Example 4 is crystallized from EtOAc/hexane. Yield 80%. HRMS (ESI) calcd for C₁₈H₁₉N₅O₃+H 354.1566, found 354.1559.

### EXAMPLE 5

### N-[4-methoxy-2-(1,3-thiazol-2-yl)phenyl]-N′-(5-methylisoxazol-3-yl)urea

To a solution of 2-iodo-4-methoxyaniline, see Lizos, D.; Tripoli, R.; Murphy, J. A. Chem. Commun. 2003, 2732-2733, (0.5 g, 2.0 mol) in 1,4-dioxane (12.5 ml) are added Pd(Ph₃P)₄ (0.231 g, 0.20 mmol) and 2-(tributylstannyl)thiazole (0.90 ml, 2.4 mmol). The reaction mixture is purged with argon and refluxed at 95° C. for 5 hr. The mixture is concentrated, diluted with Hexanes, extracted with CH₃CN, and concentrated under vacuum. 4-Methoxy-2-(1,3-thiazol-2-yl)aniline is purified by silica gel chromatography (CH₂Cl₂) to afford brown oil 0.340 g (83%). MS (ESI+) for C₁₀H₁₀N₂OS m/z 207.1 (M+H)⁺.

To a solution of the 4-methoxy-2-(1,3-thiazol-2-yl)aniline (0.17 g, 0.82 mmol) in THF (5.0 ml) are added phenyl 5-methylisoxazol-3-ylcarbamate (0.18 g, 0.82 mmol) and TEA (0.112 ml, 0.82 mmol). The reaction mixture is stirred at 50° C. for 3 hr. The solution is concentrated under vacuum and Example 5 is triturated with CH₂Cl₂/n-heptane to give a yellow solid 0.134 g (49%). HRMS (ESI) calcd for C₁₅H₁₄N₄O₃S+H 331.0865, found 331.0851.

### EXAMPLE 6

### N-[4-methoxy-2-(1,3-thiazol-2-yl)phenyl]-N′-[5-(trifluoromethyl)-1,3,4-thiadiazol-2-yl]urea

To a solution of the 4-methoxy-2-(1,3-thiazol-2-yl)aniline (0.17 g, 0.82 mmol) in THF (5.0 ml) are added 2-isocyanato-5-(trifluoromethyl)-1,3,4-thiadiazole (0.16 g, 0.82 mmol) and 4-dimethylamino pyridine (0.0005 g, 0.04 mmol). The reaction mixture is stirred at 50° C. for 3 hr. The solution is concentrated under vacuum and the residue is purified by silica gel chromatography (50% EtOAc/n-heptane) followed by the trituration with CH₂Cl₂/n-heptane to give a yellow solid 0.148 g (45%). HRMS (ESI) calcd for C₁₄H₁₀N₅O₂S₂F₃+H 402.0306, found 402.0312.

### EXAMPLE 7

### N-[4-methoxy-2-(1,3-oxazol-2-yl)phenyl]-N′-(5-methylisoxazol-3-yl)urea

To a solution of 2-iodo-4-methoxyaniline (0.6 g, 2.4 mol) in 1,4-dioxane (15.0 ml) are added Pd(Ph₃P)₄ (0.279 g, 0.24 mmol) and 2-(tributylstannyl)-1,3-oxazole (2.0 g, 5.6 mmol). The reaction mixture is purged with argon and refluxed at 95° C. for 10 hr. The mixture is concentrated, diluted with Hexanes, extracted with CH₃CN, and concentrated under vacuum. The residue is purified by silica gel chromatography (CH₂Cl₂) to afford 4-methoxy-2-(1,3-oxazol-2-yl)aniline as a brown oil 0.224 g (49%). HRMS (ESI) calcd for C₁₀H₁₀N₂O₂+H 191.0820, found 191.0813.

To a solution of the 4-methoxy-2-(1,3-oxazol-2-yl)aniline (0.109 g, 0.57 mmol) in THF (5.0 ml) are added phenyl 5-methylisoxazol-3-ylcarbamate (0.125 g, 0.57 mmol) and TEA (0.078 ml, 0.57 mmol). The reaction mixture is stirred at 50° C. for 4 hr. The solution is concentrated under vacuum and the residue is purified by silica gel chromatography (20% EtOAc/n-heptane) followed by trituration with CH₂Cl₂/n-heptane to afford Example 7 as a white solid 0.083 g (46%). HRMS (ESI) calcd for C₁₅H₁₄N₄O₄+H 315.1093, found 315.1096.

### EXAMPLE 8

### N-[4-methoxy-2-(1,3-oxazol-2-yl)phenyl]-N′-[5-(trifluoromethyl)-1,3,4-thiadiazol-2-yl]urea

To a solution of the 4-methoxy-2-(1,3-oxazol-2-yl)aniline (0.115 g, 0.6 mmol) in THF (5.0 ml) are added 2-isocyanato-5-(trifluoromethyl)-1,3,4-thiadiazole (0.118 g, 0.6 mmol) and 4-dimethylamino pyridine (0.0004 g, 0.03 mmol). The reaction mixture is stirred at 50° C. for 4 hr. The solution is concentrated under vacuum and the residue is purified by silica gel chromatography (50% EtOAc/n-heptane) followed by the trituration with CH₂Cl₂/n-heptane to afford a white solid 0.05 g (21%). HRMS (ESI) calcd for C₁₄H₁₀N₅O₃SF₃+H 386.0534, found 386.0551.

### EXAMPLE 9

### N-[2-(2-furyl)-4-methoxyphenyl]-N′-[5-(trifluoromethyl)-1,3,4-thiadiazol-2-yl]urea

To a solution of 2-iodo-4-methoxyaniline (0.42 g, 1.68 mol) in 1,4-dioxane (8.0 ml) are added Pd(Ph₃P)₄ (0.195 g, 0.168 mmol) and 2-(tributylstannyl)furan (0.63 ml, 2.0 mmol). The reaction mixture is purged with argon and refluxed at 95° C. for 3 hr. The mixture is concentrated, diluted with hexane, extracted with CH₃CN, and concentrated under vacuum. The residue is purified by silica gel chromatography (CH₂Cl₂) to afford 2-(2-furyl)-4-methoxyaniline as brown semi-solid 0.227 g (71%). HRMS (EI) calcd for C₁₁H₁₀NO₂ 189.0790, found 189.0794.

To a solution of the 2-(2-furyl)-4-methoxyaniline (0.06 g, 0.32 mmol) in THF (3.0 ml) are added 2-isocyanato-5-(trifluoromethyl)-1,3,4-thiadiazole (0.075 g, 0.384 mmol) and 4-dimethylamino pyridine (0.0002 g, 0.016 mmol). The reaction mixture is stirred at 50° C. for 3 hr. The solution is concentrated under vacuum and the residue is purified by silica gel chromatography (30% EtOAc/n-heptane) followed by the trituration with CH₂Cl₂/n-heptane to afford Example 9 as a white solid 0.04 g (33%). HRMS (ESI) calcd for C₁₅H, N₄O₃SF₃+H 385.0582, found 385.0582.

### EXAMPLE 10

### N-[2-(2-furyl)-4-methoxyphenyl]-N′-[3-(trifluoromethyl)isoxazol-5-yl]urea

To a solution of 2-(2-furyl)-4-methoxyaniline (2.4 g, 12.7 mmol) in CH₂Cl₂ (400 ml) is added dropwise, phenyl chloroformate (2.0 ml, 15.2 mmol) and pyridine (1.0 ml, 12.7 mmol) at 0° C. The reaction mixture is stirred at 0° C. for 30 min. The solution is washed with 0.1 N HCl, 5% NaHCO₃, brine, and concentrated under vacuum. The resulting solid is washed with cold EtOAc to give phenyl 2-(2-furyl)-4-methoxyphenylcarbamate as a white solid 2.63 g (67%). MS (ESI+) for C₁₈H₁₅NO₄ m/z 310.2 (M+H)⁺.

To a solution of phenyl 2-(2-furyl)-4-methoxyphenylcarbamate (0.250 g, 0.8 mmol) in THF (10 ml) are added 3-(trifluoromethyl)isoxazol-5-amine (0.121 g, 0.8 mmol) and NaH 60% dispersion in mineral oil (0.032 g, 0.8 mmol). The reaction mixture is stirred at 50° C. for 15 min. The solution is concentrated under vacuum and the residue is purified by silica gel chromatography (10% EtOAc/CH₂Cl₂) followed by trituration with CH₂Cl₂/hexanes to afford Example 10 as a white solid 0.143 g (48%). MS (ESI+) for C₁₆H₁₂F₃N₃O₄ m/z 366.3 (M+H)⁺.

### EXAMPLE 11

### N-[4-ethoxy-2-(2-furyl)phenyl]-N′-[5-(trifluoromethyl)-1,3,4-thiadiazol-2-yl]urea

To a solution of 2-bromo-4-ethoxy-1-nitrobenzene (2.0 g, 8.13 mmol) in EtOH (38 ml) and HC (1.0 ml) is added Fe (powder) (6.9 g, 121.9 mmol). The reaction mixture is refluxed at 80° C. for 1 hr. The suspension is filtered through cellulose and washed with EtOH. To this solution DOWEX 50WX2-400 ion exchange resin (16 g) is added; the mixture is allowed to spin submerged in a water bath (35-40° C.) on a rotary evaporator for 20 minutes. The mixture is filtered, and the resin washed with EtOH. The product is liberated from the resin by treatment with a solution of 20% NH₄OH/MeOH. The basic alcohol washes are concentrated in vacuo to give 2-bromo-4-ethoxyaniline as a brown oil 1.4 g (80%). MS (ESI+) for C₈H₁₀BrNO m/z 217.9 (M+H)⁺.

To a solution of 2-bromo-4-ethoxyaniline (4.3 g, 19.9 mol) in 1,4-dioxane (100 ml) are added Pd(Ph₃P)₄ (2.3 g, 1.99 mmol) and 2-(tributylstannyl)furan (7.5 ml, 23.9 mmol). The reaction mixture is refluxed at 95° C. for 3 hr. The mixture is concentrated, diluted with hexane, extracted with CH₃CN, and concentrated under vacuum. The residue is purified by silica gel chromatography (CH₂Cl₂) to afford 4-ethoxy-2-(2-furyl)aniline as a brown oil 2.5 g (63%). MS (ESI+) for C₁₂H₁₃NO₂ m/z 204.0 (M+H)⁺.

To a solution of the 4-ethoxy-2-(2-furyl)aniline (0.106 g, 0.52 mmol) in THF (5.0 ml) are added 2-isocyanato-5-(trifluoromethyl)-1,3,4-thiadiazole (0.102 g, 0.52 mmol) and NaH 60% dispersion in mineral oil (0.020 g, 0.52 mmol). The reaction mixture is stirred at 50° C. for 6 hr. The solution is concentrated under vacuum and the residue is triturated with CH₂Cl₂ to afford Example 11 as a white solid 0.086 g (42%). HRMS (ESI) calcd for C₁₆H₁₃N₄O₃SF₃+H 399.0739, found 399.0744.

### EXAMPLE 12

### N-[4-ethoxy-2-(2-furyl)phenyl]-N′-(5-methylisoxazol-3-yl)urea

To a solution of the 4-ethoxy-2-(2-furyl)aniline (0.100 g, 0.49 mmol) in THF (5.0 ml) are added phenyl 5-methylisoxazol-3-ylcarbamate (0.129 g, 0.49 mmol) and TEA (0.067 ml, 0.49 mmol). The reaction mixture is stirred at 50° C. for 6 hr. The solution is concentrated under vacuum and the residue is triturated with CH₂Cl₂ to afford a white solid 0.046 g (28%). HRMS (ESI) calcd for C₁₇H₁₇N₃O₄+H 328.1297, found 328.1295.

### EXAMPLE 13

### N-(4-methoxy-2-thien-2-ylphenyl)-N′-[5-(trifluoromethyl)-1,3,4-thiadiazol-2-yl]urea

To a solution of 2-iodo-4-methoxyaniline (0.3 g, 1.02 mol) in 1,4-dioxane (7.5 ml) are added Pd(Ph₃P)₄ (0.138 g, 0.12 mmol) and tributyl(thien-2-yl)stannane (0.46 ml, 1.45 mmol). The reaction mixture is purged with argon and refluxed at 95° C. for 6 hr. The mixture is concentrated, diluted with Hexanes, extracted with CH₃CN, and concentrated under vacuum. The residue is purified by silica gel chromatography (CH₂Cl₂) to afford 4-methoxy-2-thien-2-ylaniline as a brown oil 0.115 g (47%). MS (ESI+) for C₁₁H₁₁NOS m/z 206.1 (M+H)⁺.

To a solution of 4-methoxy-2-thien-2-ylaniline (0.09 g, 0.44 mmol) in THF (5.0 ml) are added 2-isocyanato-5-(trifluoromethyl)-1,3,4-thiadiazole (0.102 g, 0.52 mmol) and 4-dimethylamino pyridine (0.00027 g, 0.022 mmol). The reaction mixture is stirred at 50° C. for 2 hr. The solution is concentrated under vacuum and the residue is triturated with CH₂Cl₂ to afford Example 13 as a white solid 0.1 g (57%). HRMS (ESI) calcd for C₁₅H, N₄O₂S₂F₃+H 401.0354, found 401.0362.

### EXAMPLE 14

### N-[2,4-dimethoxy-5-(1,3-oxazol-2-yl)phenyl]-N′-[5-(trifluoromethyl)-1,3,4-thiadiazol-2-yl]urea

To a cooled (−65° C.) solution of oxazole (0.54 g) in THF (100 mL) is added drop-wise, a solution of 1.5 M nBuLi in pentane (5.7 mL) over a 5-minute period. The resulting solution is stirred for 35 minutes at −65° C. at which time, a solution of tributyltin chloride (2.4 mL) in THF (10 mL) is added drop-wise, and the resulting solution is allowed to warm to 0° C. Several drops of water are added, and the solvent removed in vacuo to give 2-(tributylstannyl)-1,3-oxazole that is taken up in Et₂O, washed with saturated KF, brine, dried (Na₂SO₄), and concentrated to give an oil that is carried crude.

5-Bromo-2,4-dimethoxyaniline (0.51 g), 2-(tributylstannyl)-1,3-oxazole (2.7 g), and Pd(Ph₃P)₄ (0) (0.11 g) are dissolved in dioxane (10 mL) and heated to 95° C. for 3 hours The solvent removed in vacuo to give a reside that is taken up in EtOAc, washed with saturated KF, brine, dried (Na₂SO₄), purified by silica gel chromatography to give 2,4-dimethoxy-5-(1,3-oxazol-2-yl)aniline. Yield 58%. MS (ESI+) for C₁₁H₁₂N₂O₃ m/z 221.1 (M+H)⁺.

2,4-Dimethoxy-5-(1,3-oxazol-2-yl)aniline (0.14 g), DMAP (˜10 mg), and 2-isocyanato-5-(trifluoromethyl)-1,3,4-thiadiazole (0.12 g) are suspended in 1:1 THF/DMF (10 mL) and heated to 50° C. for 4 hours, then cooled ambient temperature for an additional 12 hours. The solvents are removed in vacuo and the residue is crystallized from MeCN to give Example 14 as a white solid. Yield 34%. MS (ESI+) for C₁₅H₁₂F₃N₅O₄S m/z 416.2 (M+H)⁺.

### EXAMPLE 15

### N-[4-ethoxy-2-(2-furyl)phenyl]-N′-[3-(trifluoromethyl)isoxazol-5-yl]urea

Absolute EtOH (700 ml) is cooled in an ice bath and sodium (5.2 g) is slowly added. The cooling bath is removed and the resulting mixture allowed to stir at RT for 2 hours. 2-Bromo-4-fluoro-1-nitrobenzene (15.0 g) is slowly added, and the resulting mixture is allowed to stir for 15 hours. A solution of citric acid (1.0 M) is added until the pH is ˜4. Water (200 ml) is added, the volatiles are removed in vacuo and the residue is taken up in EtOAc, washed with water (2×100 ml) and then brine, dried (MgSO₄), and crystallized from 1-chlorobutane/n-hexane to give 2-bromo-4-ethoxy-1-nitrobenzene. Yield 88%. ¹H NMR (400 MHz, DMSO-d₆) δ 8.04, 7.40, 7.11, 4.15, 1.33.

To a solution of 2-bromo-4-ethoxy-1-nitrobenzene (2.0 g, 8.13 mmol) in EtOH (38 ml) and HCl (1.0 ml) is added Fe (powder) (6.9 g, 121.9 mmol). The reaction mixture is refluxed at 80° C. for 1 hr. The suspension is filtered through cellulose and washed with EtOH. To this solution DOWEX 50WX2-400 ion exchange resin (16 g) is added; the mixture is allowed to spin submerged in a water bath (35-40° C.) on a rotary evaporator for 20 minutes. The mixture is filtered, and the resin is washed with 3 portions of EtOH. The product is liberated from the resin by treatment with a solution of 20% NH₄OH/MeOH that is applied in 3×100 ml portions. The basic alcohol washes are concentrated in vacuo to give 2-bromo-4-ethoxyaniline as a brown oil 1.4 g (80%). HRMS (ESI) calcd for C₈H₁₀NOBr+H 216.0025, found 216.0031.

To a solution of 2-bromo-4-ethoxyaniline (4.3 g, 19.9 mol) in 1,4-dioxane (100 ml) are added Pd(Ph₃P)₄ (2.3 g, 1.99 mmol) and 2-(tributylstannyl)furan (7.5 ml, 23.8 mmol). The reaction mixture is purged with argon and refluxed at 95° C. for 2 hr. The mixture is concentrated, diluted with n-hexanes, extracted with CH₃CN, and concentrated under vacuum. The residue is purified by silica gel chromatography (CH₂Cl₂) to afford 4-ethoxy-2-(2-furyl)aniline as a brown semi-solid 2.5 g (62%). MS (ESI+) for C₁₂H₁₃NO₂ m/z 204.0 (M+H)⁺.

To a solution of 4-ethoxy-2-(2-furyl)aniline (2.5 g, 12.3 mmol) in CH₂Cl₂ (370 ml) is added dropwise, phenyl chloroformate (1.8 ml, 14.8 mmol) and pyridine (1.0 ml, 12.3 mmol) at 0° C. The reaction mixture is stirred at 0° C. for 30 min. The solution is washed with 0.1 N HCl, 5% NaHCO₃, brine, and concentrated under vacuum. The resulting solid is recrystallized from EtOAc/n-hexanes to give phenyl 4-ethoxy-2-(2-furyl)phenylcarbamate as a white solid 2.9 g (73%). HRMS (ESI) calcd for C₁₉H₁₇NO₄+H 324.1236, found 324.1246.

To a solution of 3-(trifluoromethyl)isoxazol-5-amine (0.152 g, 1.0 mmol) in THF (10 ml) is added NaH 60% dispersion in mineral oil (0.04 g, 1.0 mmol). After stirring the mixture at RT for 15 min phenyl 4-ethoxy-2-(2-furyl)phenylcarbamate (0.323 g, 1.0 mmol) is added and the reaction mixture is heated at 50° C. for 1 hour. The mixture is neutralized with 0.1M HCl, extracted with EtOAc, and the combined organic layers are dried (MgSO₄), filtered, and concentrated under vacuum. The residue is triturated with CH₂Cl₂ to afford Example 15 as a yellow solid 0.188 g (50%). HRMS (ESI) calcd for C₁₇H₁₄N₃O₄F₃+H 382.1014, found 382.1013.

### EXAMPLE 16

### N-[4-methoxy-2-(1,3-oxazol-2-yl)phenyl]-N′-[3-(trifluoromethyl)isoxazol-5-yl]urea

To a cooled (−65° C.) solution of oxazole (0.54 g) in THF (100 ml) is added dropwise, a solution of 1.5 M nBuLi in pentane (5.7 ml) over a 5-minute period. The resulting solution is stirred for 35 minutes at −65° C. at which time, a solution of tributyltin chloride (2.4 ml) in THF (10 ml) is added dropwise, and the resulting solution is allowed to warm to 0° C. Several drops of water are added, and the solvent is removed in vacuo to give a reside that is taken up in Et₂O, washed with 3×50 ml portions of saturated KF, once with brine, dried (Na₂SO₄), and concentrated to give 2-(tributylstannyl)-1,3-oxazole as an oil.

To a solution of 2-iodo-4-methoxyaniline (6.0 g, 2.4 mol) in 1,4-dioxane (110 ml) is added Pd(Ph₃P)₄ (2.8 g, 2.4 mmol) and 2-(tributylstannyl)furan (14.3 g, 40.0 mmol). The reaction mixture is purged with argon and refluxed at 95° C. for 3 hr. The mixture is concentrated, diluted with n-hexanes, extracted with CH₃CN, and concentrated under vacuum. The residue is purified by silica gel chromatography (CH₂Cl₂) to afford 4-methoxy-2-(1,3-oxazol-2-yl)aniline as a brown semi-solid 1.0 g (22%). HRMS (ESI) calcd for C₁₀H₁₀N₂O₂+H 191.0820, found 191.0813.

To a solution of 4-methoxy-2-(1,3-oxazol-2-yl)aniline (1.0 g, 5.26 mmol) in CH₂Cl₂ (160 ml) is added dropwise, phenyl chloroformate (0.8 ml, 6.3 mmol) and pyridine (0.4 ml, 5.26 mmol) at 0° C. The reaction mixture is stirred at 0° C. for 30 min. The solution is washed with 0.1 N HCl, 5% NaHCO₃, brine, and concentrated under vacuum. The resulting solid is recrystallized from EtOAc/n-hexanes to give phenyl 4-methoxy-2-(1,3-oxazol-2-yl)phenylcarbamate as a white solid 0.827 g (51%). HRMS (ESI) calcd for C₁₇H₁₄N₂O₄+H 311.1031, found 311.1038.

To a solution of 3-(trifluoromethyl)isoxazol-5-amine (0.08 g, 0.55 mmol) in DMF (10 ml) is added NaH 60% dispersion in mineral oil (0.02 g, 0.55 mmol). After stirring the mixture at RT for 15 min phenyl 4-methoxy-2-(1,3-oxazol-2-yl)phenylcarbamate (0.17 g, 055 mmol) is added and the reaction mixture is heated at 50° C. for 30 min. The mixture is neutralized with 0.1M HCl, extracted with EtOAc, and the combined organic layers are dried (MgSO₄), filtered, and concentrated under vacuum. The residue is triturated with CH₂Cl₂/n-hexanes to afford Example 16 as a white solid 0.131 g (65%). HRMS (ESI) calcd for C₁₅H, 1N₄O₄F₃+H 369.0811, found 369.0803.

### EXAMPLE 17

### N-[4-ethoxy-2-(1,3-oxazol-2-yl)phenyl]-N′-[3-(trifluoromethyl)isoxazol-5-yl]urea

To a solution of 2-bromo-4-ethoxyaniline (4.0 g, 18.5 mol) in 1,4-dioxane (80 ml) is added Pd(Ph₃P)₄ (2.1 g, 1.85 mmol) and 2-(tributylstannyl)-1,3-oxazole (21.2 g, 59.3 mmol). The reaction mixture is purged with argon and refluxed at 95° C. for 3 hr. The mixture is concentrated, diluted with n-hexanes, extracted with CH₃CN, and concentrated under vacuum. The residue is purified by silica gel chromatography (CH₂Cl₂) to afford 4-ethoxy-2-(1,3-oxazol-2-yl)aniline as a brown solid 1.65 g (45%). HRMS (ESI) calcd for C₁₁H₁₂N₂O₂+H 205.0977, found 205.0973.

To a solution of 4-ethoxy-2-(1,3-oxazol-2-yl)aniline (0.8 g, 3.9 mmol) in CH₂Cl₂ (125 ml) is added dropwise, phenyl chloroformate (0.6 ml, 4.7 mmol) and pyridine (0.3 ml, 3.9 mmol) at 0° C. The reaction mixture is stirred at 0° C. for 30 min. The solution is washed with 0.1 N HCl, 5% NaHCO₃, brine, and concentrated under vacuum. The resulting solid is recrystallized from EtOAc to give phenyl 4-ethoxy-2-(1,3-oxazol-2-yl)phenylcarbamate as an off white solid 0.88 g (69%). HRMS (ESI) calcd for C₁₈H₁₆N₂O₄+H 325.1188, found 325.1187.

To a solution of 3-(trifluoromethyl)isoxazol-5-amine (0.047 g, 0.308 mmol) in DMF (6.0 ml) is added NaH 60% dispersion in mineral oil (0.012 g, 0.308 mmol). After stirring the mixture at RT for 15 min phenyl 4-ethoxy-2-(1,3-oxazol-2-yl)phenylcarbamate (0.1 g, 0.308 mmol) is added and the reaction mixture is heated at 50° C. for 30 min. The mixture is neutralized with 0.1M HCl, extracted with EtOAc, and the combined organic layers are dried (MgSO₄), filtered, and concentrated under vacuum. The residue is purified by silica gel chromatography (40% EtOAc/heptane) followed by the trituration with CH₂Cl₂/heptane to afford Example 17 as a white solid 0.103 g (87%). HRMS (ESI) calcd for C₁₆H₁₃N₄O₄F₃+H 383.0967, found 383.0961.

### EXAMPLE 18

### N-[4-ethoxy-2-(1,3-thiazol-2-yl)phenyl]-N′-[3-(trifluoromethyl)isoxazol-5-yl]urea

To a solution of 2-bromo-4-ethoxyaniline (2.0 g, 9.2 mol) in 1,4-dioxane (40 ml) is added Pd(Ph₃P)₄ (1.0 g, 0.92 mmol) and 2-(tributylstannyl)-1,3-thiazole (4.15 g, 11.1 mmol). The reaction mixture is purged with argon and refluxed at 95° C. for 2 hr. The mixture is concentrated, diluted with n-hexanes, extracted with CH₃CN, and concentrated under vacuum. The residue is purified by silica gel chromatography (CH₂Cl₂) to afford 4-ethoxy-2-(1,3-thiazol-2-yl)aniline as a brown oil 0.809 g (40%). HRMS (ESI) calcd for C₁₁H₁₂N₂OS+H 221.0749, found 221.0745.

To a solution of 4-ethoxy-2-(1,3-thiazol-2-yl)aniline (0.77 g, 3.5 mmol) in CH₂Cl₂ (105 ml) is added dropwise, phenyl chloroformate (0.5 ml, 4.1 mmol) and pyridine (0.28 ml, 3.5 mmol) at 0° C. The reaction mixture is stirred at 0° C. for 30 min. The solution is washed with 0.1 N HCl, 5% NaHCO₃, brine, and concentrated under vacuum. The resulting solid is recrystallized from EtOAc to give phenyl 4-ethoxy-2-(1,3-thiazol-2-yl)phenylcarbamate as an off white solid 0.78 g (66%). HRMS (ESI) calcd for C₁₈H₁₆N₂O₃S+H 341.0960, found 341.0956.

To a solution of 3-(trifluoromethyl)isoxazol-5-amine (0.112 g, 0.735 mmol) in THF (5.0 ml) are added phenyl 4-ethoxy-2-(1,3-thiazol-2-yl)phenylcarbamate (0.25 g, 0.735 mmol) and TEA (0.2 ml, 1.5 mmol). The reaction mixture is stirred at 50° C. for 2 hr. Then NaH 60% dispersion in mineral oil (0.03 g, 0.735 mmol) is added and reaction mixture is stirred at RT for 15 min. The mixture is neutralized with 0.1M HCl, extracted with EtOAc, and the combined organic layers are dried (MgSO₄), filtered, and concentrated under vacuum. The residue is triturated with CH₂Cl₂/heptane to afford Example 18 as an orange solid 0.177 g (61%). HRMS (ESI) calcd for C₁₆H₁₃N₄O₃SF₃+H 399.0739, found 399.0742.

### EXAMPLE 19

### N-[4-ethoxy-2-(1,3-oxazol-2-yl)phenyl]-N′-[5-(trifluoromethyl)-1,3,4-thiadiazol-2-yl]urea

To a solution of 5-(trifluoromethyl)-1,3,4-thiadiazol-2-amine (0.13 g, 0.77 mmol) in THF (5.0 ml) are added phenyl 4-ethoxy-2-(1,3-oxazol-2-yl)phenylcarbamate (0.25 g, 0.77 mmol) and TEA (0.209 ml, 1.54 mmol). The reaction mixture is stirred at 50° C. for 2 hr. Then NaH 60% dispersion in mineral oil (0.031 g, 0.77 mmol) is added and reaction mixture is stirred at RT for 15 min. The mixture is neutralized with 0.1M HCl, extracted with EtOAc, and the combined organic layers are dried (MgSO₄), filtered, and concentrated under vacuum. The residue is purified by silica gel chromatography (10% EtOAc/CH₂Cl₂) followed by the trituration with CH₂Cl₂ to afford Example 19 as a white solid 0.098 g (32%). HRMS (ESI) calcd for C₁₅H₁₂N₅O₃SF₃+H 400.0691, found 400.0692.

### EXAMPLE 20

### N-[4-ethoxy-2-(1,3-thiazol-2-yl)phenyl]-N′-[5-(trifluoromethyl)-1,3,4-thiadiazol-2-yl]urea

To a solution of 5-(trifluoromethyl)-1,3,4-thiadiazol-2-amine (0.099 g, 0.588 mmol) in THF (5.0 ml) are added phenyl 4-ethoxy-2-(1,3-thiazol-2-yl)phenylcarbamate (0.2 g, 0.588 mmol) and TEA (0.159 ml, 1.176 mmol). The reaction mixture is stirred at 50° C. for 2 hr. Then NaH 60% dispersion in mineral oil (0.024 g, 0.588 mmol) is added and reaction mixture is stirred at RT for 15 min. The mixture is neutralized with 0.1M HCl, extracted with EtOAc, and the combined organic layers are dried (MgSO₄), filtered, and concentrated under vacuum. The residue is triturated with EtOAc/heptane to afford Example 20 as an off white solid 0.133 g (55%). HRMS (ESI) calcd for C₁₅H₁₂N₅O₂S₂F₃+H 416.0463, found 416.0469.

### EXAMPLE 21

### N-(6-cyanopyridin-3-yl)-N′-[4-ethoxy-2-(1,3-oxazol-2-yl)phenyl]urea

To a solution of 4-ethoxy-2-(1,3-oxazol-2-yl)aniline (0.204 g, 1.0 mmol) in THF (5.0 ml) are added phenyl 6-cyanopyridin-3-ylcarbamate (0.239 g, 1.0 mmol) and TEA (0.135 ml, 1.0 mmol). The reaction mixture is stirred at 50° C. for 2 hr. The formed precipitate is filtered to give Example 21 as an off white solid 0.197 g (56%). HRMS (ESI) calcd for C₁₈H₁₅N₅O₃+H 350.1253, found 350.1269.

### EXAMPLE 22

### N-[2-(1,3-oxazol-2-yl)phenyl]-N′-[3-(trifluoromethyl)isoxazol-5-yl]urea

To a solution of 2-iodoaniline (1.0 g, 4.56 mol) in 1,4-dioxane (18 ml) is added Pd(Ph₃P)₄ (0.527 g, 0.456 mmol) and 2-(tributylstannyl)-1,3-oxazole (17.8 g, 49.7 mmol). The reaction mixture is purged with argon and refluxed at 95° C. for 2 hr. The mixture is concentrated, diluted with n-hexanes, extracted with CH₃CN, and concentrated under vacuum. The residue is purified by silica gel chromatography (CH₂Cl₂) to afford 2-(1,3-oxazol-2-yl)aniline as a brown solid 0.587 g (80%). MS (ESI+) for C₉H₈N₂O m/z 161.1 (M+H)⁺.

To a solution of 3-(trifluoromethyl)isoxazol-5-amine (1.0 g, 6.57 mmol) in CH₂Cl₂ (15 ml) is added dropwise, phenyl chloroformate (1.8 ml, 14.45 mmol) and pyridine (1.0 ml, 13.14 mmol) at 0° C. The reaction mixture is stirred at 0° C. for 30 min. The reaction mixture is washed with H₂O and 1% HCl. To the combined organic layers are added pyridine (1.0 ml, 6.57 mmol), H₂O (1.0 ml), and CH₂Cl₂ (20 ml), and the mixture is stirred at RT for 3 hours. The reaction mixture is washed with 0.1N HCl and brine, dried (Na₂SO₄), and concentrated. The residue is recrystallized from n-hexanes to give phenyl 3-(trifluoromethyl)isoxazol-5-ylcarbamate as an off white solid 1.3 g (73%). MS (ESI−) for C₁₁H₇F₃N₂O₃ m/z 271.0 (M−H)⁻.

To a solution of 2-(1,3-oxazol-2-yl)aniline (0.1 g, 0.622 mmol) in THF (5.0 ml) are added phenyl 3-(trifluoromethyl)isoxazol-5-ylcarbamate (0.169 g, 0.622 mmol) and TEA (0.084 ml, 0.622 mmol). The reaction mixture is stirred at 50° C. for 3 hr. The residue is purified by silica gel chromatography (20% EtOAc/heptane) to afford Example 22 as a white solid 0.135 g (64%). MS (ESI−) for C₁₄H₉F₃N₄O₃ m/z 337.1 (M−H)⁻.

### EXAMPLE 23

### N-[2-(2-furyl)phenyl]-N′-[5-(trifluoromethyl)-1,3,4-thiadiazol-2-yl]urea

To a solution of 2-iodoaniline (1.0 g, 4.56 mol) in 1,4-dioxane (18 ml) is added Pd(Ph₃P)₄ (0.527 g, 0.456 mmol) and 2-(tributylstannyl)furan (1.7 g, 5.47 mmol). The reaction mixture is purged with argon and refluxed at 95° C. for 2 hr. The mixture is concentrated, diluted with n-hexanes, extracted with CH₃CN, and concentrated under vacuum. The residue is purified by silica gel chromatography (CH₂Cl₂) to afford 2-(2-furyl)aniline as a brown oil 0.62 g (86%). MS (ESI+) for C₁₀H₉NO m/z 160.0 (M+H)⁺.

To a solution of 2-(2-furyl)aniline (0.25 g, 1.57 mmol) in CH₂Cl₂ (40 ml) is added dropwise, phenyl chloroformate (0.236 ml, 1.88 mmol) and pyridine (0.127 ml, 1.57 mmol) at 0° C. The reaction mixture is stirred at 0° C. for 30 min. The solution is washed with 0.1 N HCl, 5% NaHCO₃, brine, and concentrated under vacuum. The resulting solid is recrystallized from EtOAc to give phenyl 2-(2-furyl)phenylcarbamate as an off white solid 0.165 g (38%). HRMS (ESI) calcd for C₁₇H₁₃NO₃+H 280.0974, found 280.0982.

To a solution of 5-(trifluoromethyl)-1,3,4-thiadiazol-2-amine (0.1 g, 0.59 mmol) in THF (5.0 ml) are added phenyl 2-(2-furyl)phenylcarbamate (0.165 g, 0.59 mmol) and TEA (0.08 ml, 0.59 mmol). The reaction mixture is stirred at 50° C. for 3 hr. The residue is purified by silica gel chromatography (20% EtOAc/heptane) to afford Example 23 as a white solid 0.77 g (85%). MS (ESI−) for C₁₄H₉F₃N₄O₂S m/z 353.0 (M−H)⁻.

### EXAMPLE 24

### N-[4-ethoxy-2-(2-furyl)phenyl]-N′-[5-(trifluoromethyl)isoxazol-3-yl]urea

3-Methyl-5-phenyl-1,2,4-oxadiazole is prepared according to literature (M. A. Perez, C. A. Dorado, J. L. Soto, Synthesis 1983, 483-6). Ethyl acetimidate hydrochloride (25.0 g, 202 mmol) is stirred in CH₂Cl₂ (400 mL) in a flask under N₂. The reaction mixture is cooled in an ice water bath and TEA (59.2 mL, 425 mmol) is added. Benzoyl chloride (23.5 mL, 202 mmol) in CH₂Cl₂ (40 mL) is added dropwise over approximately 30 minutes. After 2 hours, the reaction mixture is removed from the cooling bath and allowed to stir at RT overnight. A 1 mL aliquot of the reaction mixture is filtered, concentrated and analyzed by ¹H NMR to determine whether the reaction is complete. The reaction mixture is poured into hexane (500 mL) and the resulting mixture is filtered and concentrated. The crude product (41.0 g) is found by ¹H NMR analysis to contain ethyl N-benzoylethanimidoate (29.2 g) with the balance of the material being largely solvent. Further purification is not done. ¹H NMR (400 MHz, CDCl₃) δ 1.38, 2.06, 4.30, 7.42-7.46, 7.52-7.57, 8.01-8.03.

Hydroxylamine hydrochloride (11.7 g, 168 mmol) is suspended in dry CH₃OH (80 mL) at RT under N₂. Sodium methoxide (25 wt. % in CH₃OH) (38.4 mL, 168 mmol) is added. Crude ethyl N-benzoylethanimidoate (29.2 g, 153 mmol) is diluted with CH₃OH (88 mL) and this solution is added to the reaction mixture by canula over 20 minutes. The reaction mixture warms during the addition. The reaction mixture is stirred at RT under N₂ for 24 hours. The reaction mixture is filtered through a glass frit and the solids are carefully washed with a small volume of CH₃OH. The filtrate is concentrated and the oily residue slowly crystallizes. The crude product is recrystallized from 1:1 CH₃OH:H₂O to give 3-methyl-5-phenyl-1,2,4-oxadiazole (12.8 g, 40% yield for two steps). ¹H NMR (400 MHz, DMSO-d₆) δ 2.43, 7.62-7.65, 7.69-7.73, 8.09-8.11.

A solution of 3-methyl-5-phenyl-1,2,4-oxadiazole (2.55 g, 15.9 mmol) and iso-propyl trifluoroacetate (3.36 mL, 23.9 mmol) in anhydrous THF (16 mL) is prepared under N₂ at RT. In a second flask, a solution of diisopropylamine (5.13 mL, 36.6 mmol) in anhydrous THF (32 mL) is prepared under N₂. This solution is cooled to −40° C. and n-butyl lithium (1.61 M) (21.7 mL, 35.0 mmol) is added over 10 minutes. The solution of LDA is kept at −10° C. for 40 minutes and then it is cooled to less than −75° C. The solution of 3-methyl-5-phenyl-1,2,4-oxadiazole and iso-propyl trifluoroacetate in THF is added drop wise to the cold LDA solution over 1.25 hours using a syringe and syringe pump. After the addition of reagents is complete, the reaction mixture is maintained at less than −75° C. for 1 hour. The reaction mixture is removed from the cooling bath and allowed to warm up to near RT over the course of 1 hour. The reaction mixture is cooled to −40° C. and quenched by the addition of 1N aqueous HCl (71 mL). After quenching, the reaction mixture is concentrated to remove hexane and THF. The residue is partitioned between Et₂O (250 mL) and H₂O (250 mL). The layers are separated and the aqueous layer is extracted with Et₂O (1×150 mL, 1×100 mL). The combined organic layers are dried (MgSO₄), filtered and concentrated to yield 1,1,1-trifluoro-3-(5-phenyl-1,2,4-oxadiazol-3-yl)propane-2,2-diol (6.64 g), which is used directly in the next reaction without purification. ¹H NMR (400 MHz, DMSO-d₆) δ 3.21, 7.63-7.66, 7.70-7.74, 8.10-8.12.

Dry 1,1,1-trifluoro-3-(5-phenyl-1,2,4-oxadiazol-3-yl)propane-2,2-diol (15.9 mmol) is combined with anhydrous DMSO (15 mL) and the resulting mixture is heated at 90° C. for 2 hours. The reaction mixture is partitioned between CH₂Cl₂ (250 mL) and H₂O (250 mL). The layers are separated and the aqueous layer is extracted with CH₂Cl₂ (2×100 mL). The combined organic layers are dried (Na₂SO₄), filtered and concentrated. The crude product (5.89 g) is chromatographed (SiO₂ 300 g, eluted with 3:1 hexane:Et₂O) to give N-[5-(trifluoromethyl)isoxazol-3-yl]benzamide (3.15 g, 77% yield for two steps). ¹H NMR (400 MHz, DMSO-d₆) δ 7.54-7.58, 7.64-7.68, 7.75, 8.03-8.05, 11.8.

N-[5-(trifluoromethyl)isoxazol-3-yl]benzamide (3.09 g, 12.0 mmol) is suspended in ethylene glycol (12 mL) and the resulting mixture is warmed to 100° C. Concentrated aqueous HCl (36%, 11.6 M) (2.6 mL, 30.1 mmol) is added and the mixture is stirred for 9 hours at 100° C. The reaction mixture is cooled to RT and partitioned between CH₂Cl₂ (100 mL) and 1N NaOH (100 mL). The layers are separated and the aqueous layer is extracted with CH₂Cl₂ (3×50 mL). The combined organic layers are dried (MgSO₄), filtered and concentrated. The crude product (4.40 g) is chromatographed (SiO₂ 300 g, eluted with 2:1 Et₂O:hexane) to yield 5-trifluoromethyl-3-aminoisoxazole (1.27 g) in 69% yield. ¹H NMR (400 MHz, CDCl₃) δ 6.26.

5-Trifluoromethyl-3-aminoisoxazole (1.52 g, 10.0 mmol) is dissolved in dry CH₂Cl₂ (20 mL). Phenyl chloroformate (1.72 g, 11.0 mmol) is added. While keeping the temperature below RT, pyridine (0.79 g, 10.0 mmol) is added drop wise. The reaction mixture is washed sequentially with H₂O, 1% aqueous HCl and H₂O. The organic layer is dried (Na₂SO₄) and concentrated. The residue is recrystallized from cyclohexane to yield phenyl 5-(trifluoromethyl)isoxazol-3-ylcarbamate as colorless needles (2.56 g, 94% yield).

Example 24 is prepared from phenyl 5-(trifluoromethyl)isoxazol-3-ylcarbamate and phenyl 4-ethoxy-2-(2-furyl)phenylcarbamate (Ex 15) using the methods discussed herein.

### EXAMPLE 25

### N-[4-ethoxy-2-(1,3-oxazol-2-yl)phenyl]-N′-[5-(trifluoromethyl)isoxazol-3-yl]urea

Example 25 is prepared from phenyl 5-(trifluoromethyl)isoxazol-3-ylcarbamate and phenyl 4-ethoxy-2-(1,3-oxazol-2-yl)phenylcarbamate (Ex 17) using the methods discussed herein.

Materials and Methods for identifying binding constants:

### Assay for Positive Allosteric Modulators of α7 nAChR.

Both agonist and positive allosteric modulator activity of the α7 nAChR are assayed using a cell-based, calcium flux assay on FLIPR. SHEP-1 cells expressing a novel, mutated form of the α7 nAChR that permitted stable cell surface expression were used for these assays. The details of the mutated form of the α7 nAChR is described in WO 00/73431.

Cells were plated into each well of either a 96 or 384 well cell culture plates, they were transferred to a standard CO₂ incubator for at least 24 h to achieve confluence. The assay described below is for the 96 well assay. The 384-well assay is essentially the same, with the exception that the volumes of the reagents was reduced by a factor of 4. At confluence, the growth media was aspirated and replaced with 200 μl of new media containing a Calcium Green-1 AM to obtain a final dye concentration was 2 μM. Cells were incubated for 60 min at 37° C., then washed 4 times leaving 100 μl of assay buffer in each well. The details of the assay buffer were described in WO 00/73431. At this point, the cell culture plate containing the cells loaded with the calcium indicator dye was placed in FLIPR. FLIPR was configured to excite the Calcium Green at 488 nm and emission was read using a 520 nm filter set.

Compounds were prepared as a solutions in an assay buffer. The assay was initiated by collecting 10 baseline data points at 1.5 second intervals. After the baseline points were collected, 100 μl of compound was added to the well. The resulting 1:1 dilution achieved a final concentration 30 μM for each compound. An additional 3 min of data was collected. After 3 min measurements, acetylcholine was added at a final concentration of 100 μM. Acetylcholine produced a reproducible rapid and transient calcium flux. Positive allosteric modulator activity was defined as a compound that increased the acetylcholine response by greater than 4 standard deviations of the mean response. The examples prepared herein had activity between 10 nM and 10 μM.

1. A compound of Formula I: 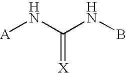
wherein X is O or S;
A is 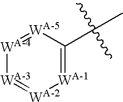
wherein each W^{A-1}, W^{A-2}, W^{A-3}, W^{A-4}, and W^{A-5} are independently N or CR_{A}, provided that no more than four of W^{A-1}, W^{A-2}, W^{A-3}, W^{A-4}, or W^{A-5} are simultaneously N;
Each R_{A} is R_{A-1} or R_{A-2}, provided that one R_{A} is R_{A-2};
Each R_{A-1} is independently H, halogen, alkyl, haloalkyl, substituted alkyl, alkenyl, haloalkenyl, substituted alkenyl, alkynyl, haloalkynyl, substituted alkynyl, heterocycloalkyl, haloheterocycloalkyl, substituted heterocycloalkyl, cycloalkyl, halocycloalkyl, substituted cycloalkyl, aryl, —N₃, —SCN, —CN, —NO₂, —OR₇, —SR₈, —S(O)R₈, —S(O)₂R₈, —N(R₉)₂, —C(O)R₁₀, —C(O)OR₇, —C(O)N(R₉)₂, —NR₉C(O)R₁₀, —C(R₁₀)═NOR₇, —S(O)₂N(R₉)₂, —NR₉S(O)₂R₈, —N(R₉)C(O)N(R₉)₂;
R_{A-2} is R₁, R₂, OR₁, OR₂, N(R_{A-3})R₁, N(R_{A-3})R₂, SR₁, and SR₂;
R_{A-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
B is a five or six-membered aromatic ring having up to 4 heteroatoms selected from —O—, —N(R_{B-3})—, ═N—, or —S—;
wherein B is 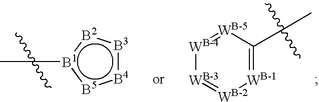
B¹ is N, or C;
B², B³, B⁴, and B⁵ are independently N, O, S, C, provided that when valency allows, the N can have a third bond to R_{B-3}, and further provided that when valency allows, the C can have a fourth bond to R_{B-1};
Each R_{B-1} is independently H, halogen, alkyl, haloalkyl, substituted alkyl, cycloalkyl, halocycloalkyl, substituted cycloalkyl, alkenyl, haloalkenyl, substituted alkenyl, alkynyl, haloalkynyl, substituted alkynyl, heterocycloalkyl, haloheterocycloalkyl, substituted heterocycloalkyl, aryl, —CN, —N₃, —NO₂, —COR₁₀, —CO₂R₇, —CON(R₉)₂, —C(R₁₀)═NOR₇, —SCN, —OR₇, —N(R₉)₂, —SR₈, —SOR₈, —SO₂R₈, —SN(R₉)₂, —SON(R₉)₂, —SO₂N(R₉)₂; or
when two R_{B-1} are on adjacent carbon atoms, the two R_{B-1} may combine to form a 5-7-membered ring fused to the 5 or 6 membered ring giving a fused-bicyclic-ring system; wherein the 5-7-membered ring is saturated or unsaturated having up to two heteroatoms selected from —O—, —S—, —N(R_{B-3})—, or —N═ and further having substitution where valency allows on the 5-7-membered ring with up to 2 substitutents independently selected from R_{B-2};
Each R_{B-2} is independently H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, —CN, —NO₂, —OR₇, —SR₈, —S(O)₂R₈, —S(O)R₈, —OS(O)₂R₈, —N(R₉)₂, —C(O)R₁₀, —C(S)R₁₀, —C(O)₂R₇, —C(O)N(R₉)₂, —NR₉C(O)R₁₀, —S(O)₂N(R₉)₂, —NR₉S(O)₂R₈, —N(R₉)C(O)N(R₉)₂, or aryl;
R_{B-3} is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
Each W^{B-1}, W^{B-2}, W^{B-3}, W^{B-4}, and W^{B-5} are independently N or CR_{B-1}, provided that no more than 4 of W^{B-1}, W^{B-2}, W^{B-3}, W^{B-4}, or W^{B-5} are simultaneously N;
R₁ is a 5-membered heteroaromatic mono-cyclic moiety containing within the ring 1-3 heteroatoms independently selected from the group consisting of ═N—, —N(R_{1-N})—, —O—, and —S—, and having 0-2 substituent selected from R₁₋₁, and further having 0-4 substituents independently selected from F, Cl, Br, or I;
or R₁ is a 9-membered fused-ring moiety having a 6-membered ring fused to a 5-membered ring including the formula 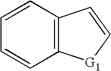
wherein G₁ is O, S or NR_{1-N}, 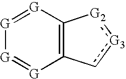
wherein each G is independently CH, C(R_{1-C}), or N, and each G₂ and G₃ are independently selected from CH₂, CH, C(R_{1-C}), O, S, N, and N(R_{1-N}), provided that both G₂ and G₃ are not simultaneously O, simultaneously S, or simultaneously O and S, or 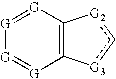
wherein each G is independently CH, C(R_{1-C}), or N, and each G₂ and G₃ are independently selected from CH₂, CH, C(R_{1-C}), O, S, N, and N(R_{1-N}), provided that each 9-membered fused-ring moiety has 0-1 substituent selected from R₁₋₁, and further having 0-3 substituents independently selected from F, Cl, Br, or I, wherein the R₁ moiety attaches to other substituents as defined in formula I at any position as valency allows;
Each R_{1-C} is independently a bond, R₁₋₁, F, Cl, Br, or I, provided that there is only one bond and further provided that R₁ can have only up to one substituent from R₁₋₁, and up to 3 substituents from halogen;
R_{1-N} is H, alkyl, haloalkyl, substituted alkyl, cycloalkyl, halocycloalkyl, substituted cycloalkyl, heterocycloalkyl, haloheterocycloalkyl, or substituted heterocycloalkyl;
R₁₋₁ is alkyl, substituted alkyl, haloalkyl, —OR₁₋₂, —SR₁₋₂, —CN, —NO₂, —N(R₁₋₃)₂;
Each R₁₋₂ is independently H, alkyl, cycloalkyl, heterocycloalkyl, haloalkyl, halocycloalkyl, or haloheterocycloalkyl;
Each R₁₋₃ is independently H, alkyl, cycloalkyl, heterocycloalkyl, haloalkyl, halocycloalkyl, or haloheterocycloalkyl;
R₂ is a 6-membered heteroaromatic mono-cyclic moiety containing within the ring 1-4 heteroatoms selected from ═N— and having 0-1 substituent selected from R₂₋₁ and 0-3 substituent(s) independently selected from F, Cl, Br, or I;
or R₂ is 10-membered heteroaromatic bi-cyclic moieties containing within one or both rings 1-3 heteroatoms selected from ═N—, each 10-membered fused-ring moiety having 0-1 substituent selected from R₂₋₁ and 0-3 substituent(s) independently selected from F, Cl, Br, or I, wherein the R₂ moiety attaches to other substituents as defined in formula I at any position as valency allows;
R₂₋₁ is alkyl, substituted alkyl, haloalkyl, —OR₂₋₂, —SR₂₋₂, —CN, —NO₂, —N(R₂₋₃)₂;
Each R₂₋₂ is independently H, alkyl, cycloalkyl, heterocycloalkyl, haloalkyl, halocycloalkyl, or haloheterocycloalkyl;
Each R₂₋₃ is independently H, alkyl, cycloalkyl, heterocycloalkyl, haloalkyl, halocycloalkyl, or haloheterocycloalkyl;
R₇ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
R₈ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
Each R₉ is independently H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
R₁₀ is H, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, or aryl;
or pharmaceutical composition, pharmaceutically acceptable salt, racemic mixture, or pure enantiomer thereof.

2. The compound of claim 1, wherein X is O.

3. The compound of claim 2, wherein W^{A-1}, W^{A-2}, W^{A-3}, W^{A-4}, and W^{A-5} are each CR_{A}.

4. The compound of claim 3, wherein W^{A-1} and W^{A-4} are CH; W^{A-2} is CH or CR_{A-1}, where R_{A-1} is halo; W^{A-3} is CR_{A-1}; and W^{A-5} is CR_{A-2}.

5. The compound of claim 4, wherein B is thienyl, thiazolyl, furanyl, isothiazolyl, thiadiazolyl, isoxazolyl, oxazolyl, and pyridinyl, any of which is optionally substituted as allowed by formula I.

6. The compound of claim 5, wherein R_{A-1} of W^{A-3} is OR₇.

7. The compound of claim 6, wherein R_{A-2} is R₁, OR₁, NHR₁, R₂, OR₂, and NHR₂.

8. The compound of claim 7, wherein R₇ is alkyl, and substituted alkyl;
wherein R₁ is independently any one of thienyl, thiazolyl, furanyl, isothiazolyl, thiadiazolyl, isoxazolyl, and oxazolyl, any of which is optionally substituted as allowed by formula I;
and wherein R₂ is pyridinyl, any of which is optionally substituted as allowed by formula I.

9. The compound of claim 8, wherein B is isoxazol-3-yl having a substituent at C-5.

10. The compound of claim 9, wherein the compound is
N-[4-ethoxy-2-(pyridin-4-ylamino)phenyl]-N′-(5-methylisoxazol-3-yl)urea;
N-[4-ethoxy-2-(pyridin-3-ylamino)phenyl]-N′-(5-methylisoxazol-3-yl)urea;
N-[4-ethoxy-2-(pyridin-2-ylamino)phenyl]-N′-(5-methylisoxazol-3-yl)urea;
and pharmaceutically acceptable salts thereof.

11. The compound of claim 9, wherein the compound is
N-[4-methoxy-2-(1,3-oxazol-2-yl)phenyl]-N′-(5-methylisoxazol-3-yl)urea;
N-[4-ethoxy-2-(1,3-oxazol-2-yl)phenyl]-N′-[5-(trifluoromethyl)isoxazol-3-yl]urea;
and pharmaceutically acceptable salts thereof.

12. The compound of claim 9, wherein the compound is
N-[4-methoxy-2-(1,3-thiazol-2-yl)phenyl]-N′-(5-methylisoxazol-3-yl)urea;
and pharmaceutically acceptable salts thereof.

13. The compound of claim 9, wherein the compound is
N-[4-ethoxy-2-(2-furyl)phenyl]-N′-(5-methylisoxazol-3-yl)urea;
N-[4-ethoxy-2-(2-furyl)phenyl]-N′-[5-(trifluoromethyl)isoxazol-3-yl]urea;
and pharmaceutically acceptable salts thereof.

14. The compound of claim 9, wherein the compound is
N-[4-ethoxy-5-fluoro-2-(pyridin-4-ylamino)phenyl]-N′-(5-methylisoxazol-3-yl)urea;
N-[4-ethoxy-5-fluoro-2-(pyridin-3-ylamino)phenyl]-N′-(5-methylisoxazol-3-yl)urea;
N-[4-ethoxy-5-fluoro-2-(pyridin-2-ylamino)phenyl]-N′-(5-methylisoxazol-3-yl)urea;
N-[5-chloro-4-ethoxy-2-(pyridin-4-ylamino)phenyl]-N′-(5-methylisoxazol-3-yl)urea;
N-[5-chloro-4-ethoxy-2-(pyridin-3-ylamino)phenyl]-N′-(5-methylisoxazol-3-yl)urea;
N-[5-chloro-4-ethoxy-2-(pyridin-2-ylamino)phenyl]-N′-(5 methylisoxazol-3-yl)urea;
N-[4-(2-methoxy-ethoxy)-2-(pyridin-4-ylamino)phenyl]-N′-(5-methylisoxazol-3-yl)urea;
N-[4-(2-methoxy-ethoxy)-2-(pyridin-3-ylamino)phenyl]-N′-(5-methylisoxazol-3-yl)urea;
N-[4-(2-methoxy-ethoxy)-2-(pyridin-2-ylamino)phenyl]-N′-(5-methylisoxazol-3-yl)urea;
N-[5-fluoro-4-(2-methoxy-ethoxy)-2-(pyridin-4-ylamino)phenyl]-N′-(5-methylisoxazol-3-yl)urea;
N-[5-fluoro-4-(2-methoxy-ethoxy)-2-(pyridin-3-ylamino)phenyl]-N′-(5-methylisoxazol-3-yl)urea;
N-[5-fluoro-4-(2-methoxy-ethoxy)-2-(pyridin-2-ylamino)phenyl]-N′-(5-methylisoxazol-3-yl)urea; and pharmaceutically acceptable salts thereof.

15. The compound of claim 9, wherein the compound is
N-[4-methoxy-5-fluoro-2-(1,3-thiazol-2-yl)phenyl]-N′-(5-methylisoxazol-3-yl)urea;
N-[5-chloro-4-methoxy-2-(1,3-thiazol-2-yl)phenyl]-N′-(5-methylisoxazol-3-yl)urea;
and pharmaceutically acceptable salts thereof.

16. The compound of claim 9, wherein the compound is
N-[4-methoxy-5-fluoro-2-(1,3-oxazol-2-yl)phenyl]-N′-(5-methylisoxazol-3-yl)urea;
N-[5-chloro-4-methoxy-2-(1,3-oxazol-2-yl)phenyl]-N′-(5-methylisoxazol-3-yl)urea;
N-[4-ethoxy-5-fluoro-2-(1,3-oxazol-2-yl)phenyl]-N′-[5-(trifluoromethyl)isoxazol-3-yl]urea;
N-[5-chloro-4-ethoxy-2-(1,3-oxazol-2-yl)phenyl]-N′-[5-(trifluoromethyl)isoxazol-3-yl]urea;
N-[4-(2-methoxy-ethoxy)-2-(1,3-oxazol-2-yl)phenyl]-N′-[5-(trifluoromethyl)isoxazol-3-yl]urea;
N-[5-fluoro-4-(2-methoxy-ethoxy)-2-(1,3-oxazol-2-yl)phenyl]-N′-[5-(trifluoromethyl)isoxazol-3-yl]urea; and pharmaceutically acceptable salts thereof.

17. The compound of claim 9, wherein the compound is
N-[4-ethoxy-5-fluoro-2-(2-furyl)phenyl]-N′-(5-methylisoxazol-3-yl)urea;
N-[4-ethoxy-5-fluoro-2-(2-furyl)phenyl]-N′-[5-(trifluoromethyl)isoxazol-3-yl]urea;
N-[5-chloro-4-ethoxy-2-(2-furyl)phenyl]-N′-(5-methylisoxazol-3-yl)urea;
N-[5-chloro-4-ethoxy-2-(2-furyl)phenyl]-N′-[5-(trifluoromethyl)isoxazol-3-yl]urea;
N-[4-(2-methoxy-ethoxy)-2-(2-furyl)phenyl]-N′-(5-methylisoxazol-3-yl)urea;
N-[4-(2-methoxy-ethoxy)-2-(2-furyl)phenyl]-N′-[5-(trifluoromethyl)isoxazol-3-yl]urea;
N-[5-fluoro-4-(2-methoxy-ethoxy)-2-(2-furyl)phenyl]-N′-(5-methylisoxazol-3-yl)urea;
N-[5-fluoro-4-(2-methoxy-ethoxy)-2-(2-furyl)phenyl]-N′-[5-(trifluoromethyl)isoxazol-3-yl]urea; and pharmaceutically acceptable salts thereof.

18. The compound of claim 8, wherein B is isoxazol-5-yl having a substituent at C-3.

19. The compound of claim 18, wherein the compound is
N-[2-(2-furyl)-4-methoxyphenyl]-N′-[3-(trifluoromethyl)isoxazol-5-yl]urea;
N-[4-ethoxy-2-(2-furyl)phenyl]-N′-[3-(trifluoromethyl)isoxazol-5-yl]urea; and pharmaceutically acceptable salts thereof.

20. The compound of claim 18, wherein the compound is
N-[4-(2-methoxy-ethoxy)-2-(2-furyl)phenyl]-N′-[3-(trifluoromethyl)isoxazol-5-yl]urea;
N-[5-fluoro-2-(2-furyl)-4-methoxyphenyl]-N′-[3-(trifluoromethyl)isoxazol-5-yl]urea;
N-[4-ethoxy-5-fluoro-2-(2-furyl)phenyl]-N′-[3-(trifluoromethyl)isoxazol-5-yl]urea;
N-[5-chloro-2-(2-furyl)-4-methoxyphenyl]-N′-[3-(trifluoromethyl)isoxazol-5-yl]urea;
N-[5-chloro-4-ethoxy-2-(2-furyl)phenyl]-N′-[3-(trifluoromethyl)isoxazol-5-yl]urea;
N-[5-fluoro-4-(2-methoxy-ethoxy)-2-(2-furyl)phenyl]-N′-[3-(trifluoromethyl)isoxazol-5-yl]urea; and pharmaceutically acceptable salts thereof.

21. The compound of claim 18, wherein the compound is
N-[4-methoxy-2-(1,3-oxazol-2-yl)phenyl]-N′-[3-(trifluoromethyl)isoxazol-5-yl]urea;
N-[4-ethoxy-2-(1,3-oxazol-2-yl)phenyl]-N′-[3-(trifluoromethyl)isoxazol-5-yl]urea;
N-[2-(1,3-oxazol-2-yl)phenyl]-N′-[3-(trifluoromethyl)isoxazol-5-yl]urea; and pharmaceutically acceptable salts thereof.

22. The compound of claim 18, wherein the compound is
N-[4-methoxy-5-fluoro-2-(1,3-oxazol-2-yl)phenyl]-N′-[3-(trifluoromethyl)isoxazol-5-yl]urea;
N-[4-ethoxy-5-fluoro-2-(1,3-oxazol-2-yl)phenyl]-N′-[3-(trifluoromethyl)isoxazol-5-yl]urea;
N-[5-fluoro-2-(1,3-oxazol-2-yl)phenyl]-N′-[3-(trifluoromethyl)isoxazol-5-yl]urea;
N-[5-chloro-4-methoxy-2-(1,3-oxazol-2-yl)phenyl]-N′-[3-(trifluoromethyl)isoxazol-5-yl]urea;
N-[5-chloro-4-ethoxy-2-(1,3-oxazol-2-yl)phenyl]-N′-[3-(trifluoromethyl)isoxazol-5-yl]urea;
N-[5-chloro-2-(1,3-oxazol-2-yl)phenyl]-N′-[3-(trifluoromethyl)isoxazol-5-yl]urea;
N-[4-(2-methoxy-ethoxy)-2-(1,3-oxazol-2-yl)phenyl]-N′-[3-(trifluoromethyl)isoxazol-5-yl]urea;
N-[5-fluoro-4-(2-methoxy-ethoxy)-2-(1,3-oxazol-2-yl)phenyl]-N′-[3-(trifluoromethyl)isoxazol-5-yl]urea;
N-[5-fluoro-4-(2-methoxy-ethoxy)-2-(1,3-thiazol-2-yl)phenyl]-N′-[3-(trifluoromethyl)isoxazol-5-yl]urea; and pharmaceutically acceptable salts thereof.

23. The compound of claim 18, wherein the compound is
N-[4-ethoxy-2-(1,3-thiazol-2-yl)phenyl]-N′-[3-(trifluoromethyl)isoxazol-5-yl]urea;
and pharmaceutically acceptable salts thereof.

24. The compound of claim 18, wherein the compound is
N-[4-ethoxy-5-fluoro-2-(1,3-thiazol-2-yl)phenyl]-N′-[3-(trifluoromethyl)isoxazol-5-yl]urea;
N-[5-chloro-4-ethoxy-2-(1,3-thiazol-2-yl)phenyl]-N′-[3-(trifluoromethyl)isoxazol-5-yl]urea;
N-[4-(2-methoxy-ethoxy)-2-(1,3-thiazol-2-yl)phenyl]-N′-[3-(trifluoromethyl)isoxazol-5-yl]urea; and pharmaceutically acceptable salts thereof.

25. The compound of claim 8, wherein B is 1,3,4-thiadiazol-2-yl having substitution at C5.

26. The compound of claim 25, wherein the compound is
N-[4-ethoxy-2-(1,3-thiazol-2-yl)phenyl]-N′-[5-(trifluoromethyl)-1,3,4-thiadiazol-2-yl]urea;
N-[4-methoxy-2-(1,3-thiazol-2-yl)phenyl]-N′-[5-(trifluoromethyl)-1,3,4-thiadiazol-2-yl]urea; and pharmaceutically acceptable salts thereof.

27. The compound of claim 25, wherein the compound is
N-[4-methoxy-5-fluoro-2-(1,3-thiazol-2-yl)phenyl]-N′-[5-(trifluoromethyl)-1,3,4-thiadiazol-2-yl]urea;
N-[4-ethoxy-5-fluoro-2-(1,3-thiazol-2-yl)phenyl]-N′-[5-(trifluoromethyl)-1,3,4-thiadiazol-2-yl]urea;
N-[5-chloro-4-methoxy-2-(1,3-thiazol-2-yl)phenyl]-N′-[5-(trifluoromethyl)-1,3,4-thiadiazol-2-yl]urea;
N-[5-chloro-4-ethoxy-2-(1,3-thiazol-2-yl)phenyl]-N′-[5-(trifluoromethyl)-1,3,4-thiadiazol-2-yl]urea;
N-[4-(2-methoxy-ethoxy)-2-(1,3-thiazol-2-yl)phenyl]-N′-[5-(trifluoromethyl)-1,3,4-thiadiazol-2-yl]urea;
N-[5-fluoro-4-(2-methoxy-ethoxy)-2-(1,3-thiazol-2-yl)phenyl]-N′-[5-(trifluoromethyl)-1,3,4-thiadiazol-2-yl]urea; and pharmaceutically acceptable salts thereof.

28. The compound of claim 25, wherein the compound is
N-[2,4-dimethoxy-5-(1,3-oxazol-2-yl)phenyl]-N′-[5-(trifluoromethyl)-1,3,4-thiadiazol-2-yl]urea;
N-[4-ethoxy-2-(1,3-oxazol-2-yl)phenyl]-N′-[5-(trifluoromethyl)-1,3,4-thiadiazol-2-yl]urea;
N-[4-methoxy-2-(1,3-oxazol-2-yl)phenyl]-N′-[5-(trifluoromethyl)-1,3,4-thiadiazol-2-yl]urea; and pharmaceutically acceptable salts thereof.

29. The compound of claim 25, wherein the compound is
N-[4-methoxy-5-fluoro-2-(1,3-oxazol-2-yl)phenyl]-N′-[5-(trifluoromethyl)-1,3,4-thiadiazol-2-yl]urea;
N-[4-ethoxy-5-fluoro-2-(1,3-oxazol-2-yl)phenyl]-N′-[5-(trifluoromethyl)-1,3,4-thiadiazol-2-yl]urea;
N-[5-chloro-4-methoxy-2-(1,3-oxazol-2-yl)phenyl]-N′-[5-(trifluoromethyl)-1,3,4-thiadiazol-2-yl]urea;
N-[5-chloro-4-ethoxy-2-(1,3-oxazol-2-yl)phenyl]-N′-[5-(trifluoromethyl)-1,3,4-thiadiazol-2-yl]urea;
N-[4-(2-methoxy-ethoxy)-2-(1,3-oxazol-2-yl)phenyl]-N′-[5-(trifluoromethyl)-1,3,4-thiadiazol-2-yl]urea;
N-[5-fluoro-4-(2-methoxy-ethoxy)-2-(1,3-oxazol-2-yl)phenyl]-N′-[5-(trifluoromethyl)-1,3,4-thiadiazol-2-yl]urea; and pharmaceutically acceptable salts thereof.

30. The compound of claim 25, wherein the compound is
N-[2-(2-furyl)phenyl]-N′-[5-(trifluoromethyl)-1,3,4-thiadiazol-2-yl]urea;
N-[2-(2-furyl)-4-methoxyphenyl]-N′-[5-(trifluoromethyl)-1,3,4-thiadiazol-2-yl]urea;
N-[4-ethoxy-2-(2-furyl)phenyl]-N′-[5-(trifluoromethyl)-1,3,4-thiadiazol-2-yl]urea; and pharmaceutically acceptable salts thereof.

31. The compound of claim 25, wherein the compound is
N-[5-fluoro-2-(2-furyl)-4-methoxyphenyl]-N′-[5-(trifluoromethyl)-1,3,4-thiadiazol-2-yl]urea;
N-[4-ethoxy-5-fluoro-2-(2-furyl)phenyl]-N′-[5-(trifluoromethyl)-1,3,4-thiadiazol-2-yl]urea;
N-[5-fluoro-2-(2-furyl)phenyl]-N′-[5-(trifluoromethyl)-1,3,4-thiadiazol-2-yl]urea;
N-[5-chloro-2-(2-furyl)-4-methoxyphenyl]-N′-[5-(trifluoromethyl)-1,3,4-thiadiazol-2-yl]urea;
N-[5-chloro-4-ethoxy-2-(2-furyl)phenyl]-N′-[5-(trifluoromethyl)-1,3,4-thiadiazol-2-yl]urea;
N-[5-chloro-2-(2-furyl)phenyl]-N′-[5-(trifluoromethyl)-1,3,4-thiadiazol-2-yl]urea;
N-[4-(2-methoxy-ethoxy)-2-(2-furyl)phenyl]-N′-[5-(trifluoromethyl)-1,3,4-thiadiazol-2-yl]urea;
N-[5-fluoro-4-(2-methoxy-ethoxy)-2-(2-furyl)phenyl]-N′-[5-(trifluoromethyl)-1,3,4-thiadiazol-2-yl]urea; and pharmaceutically acceptable salts thereof.

32. The compound of claim 25, wherein the compound is N-(4-methoxy-2-thien-2-ylphenyl)-N′-[5-(trifluoromethyl)-1,3,4-thiadiazol-2-yl]urea; and pharmaceutically acceptable salts thereof.

33. The compound of claim 25, wherein the compound is
N-(5-fluoro-4-methoxy-2-thien-2-ylphenyl)-N′-[5-(trifluoromethyl)-1,3,4-thiadiazol-2-yl]urea;
N-(5-chloro-4-methoxy-2-thien-2-ylphenyl)-N′-[5-(trifluoromethyl)-1,3,4-thiadiazol-2-yl]urea; and pharmaceutically acceptable salts thereof.

34. The compound of claim 25, wherein the compound is N-[4-ethoxy-2-(pyridin-3-ylamino)phenyl]-N′-[5-(trifluoromethyl)-1,3,4-thiadiazol-2-yl]urea; and pharmaceutically acceptable salts thereof.

35. The compound of claim 25, wherein the compound is
N-[4-ethoxy-5-fluoro-2-(pyridin-3-ylamino)phenyl]-N′-[5-(trifluoromethyl)-1,3,4-thiadiazol-2-yl]urea;
N-[5-chloro-4-ethoxy-2-(pyridin-3-ylamino)phenyl]-N′-[5-(trifluoromethyl)-1,3,4-thiadiazol-2-yl]urea;
N-[4-(2-methoxy-ethoxy)-2-(pyridin-3-ylamino)phenyl]-N′-[5-(trifluoromethyl)-1,3,4-thiadiazol-2-yl]urea;
N-[5-fluoro-4-(2-methoxy-ethoxy)-2-(pyridin-3-ylamino)phenyl]-N′-[5-(trifluoromethyl)-1,3,4-thiadiazol-2-yl]urea; and pharmaceutically acceptable salts thereof.

36. The compound of claim 8, wherein B is pyridinyl.

37. The compound of claim 36, wherein the compound is N-(6-cyanopyridin-3-yl)-N′-[4-ethoxy-2-(1,3-oxazol-2-yl)phenyl]urea, and pharmaceutically acceptable salts thereof.

38. The compound of claim 36, wherein the compound is
N-(6-cyanopyridin-3-yl)-N′-[4-ethoxy-5-fluoro-2-(1,3-oxazol-2-yl)phenyl]urea;
N-(6-cyanopyridin-3-yl)-N′-[5-chloro-4-ethoxy-2-(1,3-oxazol-2-yl)phenyl]urea;
N-(6-cyanopyridin-3-yl)-N′-[4-(2-methoxy-ethoxy)-2-(1,3-oxazol-2-yl)phenyl]urea;
N-(6-cyanopyridin-3-yl)-N′-[5-fluoro-4-(2-methoxy-ethoxy)-2-(1,3-oxazol-2-yl)phenyl]urea; and pharmaceutically acceptable salts thereof.

39. A compound of claim 1, wherein the compound has an isotopic label.

40. A compound of claim 1, wherein the compound contains a photoaffinity label wherein the compound becomes irreversibly incorporated into the nAChR upon exposure to ultraviolet light.

41. A pharmaceutical composition comprising a compound of claim 1, optionally comprising another agent including an anti-psychotic agent;
an agent that increases the level of ACh in the brain;
an agent that increases ACh levels, inhibits the activity of acetylcholinesterase, or activates the production of ACh;
a monoamine reuptake inhibitor;
a psychostimulant; or
an agent that is an alpha 7 nAChR agonist.

42. A method for treating a disease or condition in a mammal in need thereof, wherein the mammal receives symptomatic relief from activation of an alpha 7 nAChR comprising the administration of a therapeutically effective amount of a compound of claim 1.

43. The method of claim 42, wherein the disease or condition is cognitive and attention deficit symptoms of Alzheimer's, neurodegeneration associated with diseases such as Alzheimer's disease, pre-senile dementia (mild cognitive impairment), senile dementia, schizophrenia or psychosis and related cognitive deficits associated therewith, attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependant drug cessation, Gilles de la Tourette's Syndrome, glaucoma, neurodegeneration associated with glaucoma, or symptoms associated with pain.

44. The method of claim 42, wherein the disease or condition is attention deficit hyperactivity disorder and wherein the mammal receives symptomatic relief from the administration of at least one of a monoamine reuptake inhibitor, or psychostimulant for a therapeutically effective interval, optionally wherein the psychostimulant is methylphenidate (Ritalin) administered at about 0.01 to about 0.85 mg/kg/day; dextroamphetamine (Dexedrine) administered at about 0.07 to about 0.85 mg/kg/day; amphetamine (Adderall) administered at about 0.05 to about 0.6 mg/kg/day; and pemoline (Cylert) administered at about 0.1 to about 1.6 mg/kg/day; and wherein the monoamine reuptake inhibitor is desipramine (Norpramin) administered at about 0.5 to about 5.0 mg/kg/day; nortriptyline administered at about 0.1 to about 3.0 mg/kg/day; atomoxetine (Strattera) administered at about 0.1 to about 3.0 mg/kg/day; reboxetine administered at about 0.03 to about 3.0 mg/kg/day; fluoxetine (Prozac) at about 0.2 to about 20 mg/kg/day; tomoxetine administered at about at about 0.1 to about 1.1 mg/kg/day; bupropion (Wellbutrin) administered at about at about 1.0 to about 1.1 mg/kg/day; and modaphonil (Provigil) administered at about at about 1.0 to about 5.7 mg/kg/day.

45. The method of claim 44, wherein the mammal receives therapeutic relief from the administration of an agent that inhibits the activity of acetylcholinesterase; wherein the agent inhibiting acetylcholinesterase is optionally Aricept and Reminyl.

46. The method of claim 44, wherein the mammal receives therapeutic relief from the administration of an agent that is ACh or that increases levels of ACh in the brain, optionally ACh or a nutritional supplement.

47. A method for treating a disease or condition in a mammal in need thereof, wherein the mammal receives symptomatic relief from decreasing the level of TNF-α comprising administration of a therapeutically effective amount of a compound of claim 1.

48. The method of claim 47, wherein the symptomatic relief would be to treat the mammal for pain, inflammation, cancer, or diabetes.

49. A method for treating a disease or condition in a mammal in need thereof, wherein the mammal receives symptomatic relief from increasing vascular angiogensis, optionally wherein the disease or condition is wound healing, healing bone fracture, ischemic heart disease, or stable angina pectoris, comprising administering a therapeutically effective amount of a compound of claim 1.

50. A method for diagnosing disease in a mammal, comprising administering a compound of claim 39 to the mammal and detecting the binding of that compound to an alpha 7 nAChR, optionally using position emission topography or single-photon emission computed tomography.

51. The method of claim 50, wherein the disease is Alzheimer's disease, neurodegeneration associated with diseases such as Alzheimer's disease, pre-senile dementia (mild cognitive impairment), senile dementia, Parkinson's disease, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, diabetic retinopathy, or symptoms associated with pain.
